(19) United States
(12) Patent Application Publication
(10) Pub. No.: US 2017/0253673 A1
(43) Pub. Date: Sep. 7, 2017

(54) STIMULI-RESPONSIVE PROTEIN-POLYMER CONJUGATES FOR BIOSEPARATION

(71) Applicant: BIOASTRA TECHNOLOGIES INC., Montreal (CA)

(72) Inventors: Christopher KENT, Montreal (CA); Wilms BAILLE, Laval (CA); Marc GAUTHIER, St-Bruno-de-Montarville (CA); Yi ZHAO, Longueuil (CA); Nicolas COTTENYE, Montreal (CA)

(21) Appl. No.: 15/515,936

(22) PCT Filed: Sep. 30, 2015
(86) PCT No.: PCT/CA2015/050979

§ 371 Date: Mar. 30, 2017

Related U.S. Application Data
(60) Provisional application No. 62/057,642, filed on Sep. 30, 2014.

(51) Int. Cl. C07K 17/08; C08G 81/02; C07K 1/32; C07K 16/06; B01D 15/38; C07K 1/22; C07K 1/34; C07K 1/36; B01D 15/36; C07K 14/31; C07K 1/113

(52) U.S. Cl. CPC C07K 17/08; C07K 14/31; C08G 81/02; C07K 1/32; C07K 16/065; C07K 1/113; C07K 1/22; C07K 1/34; C07K 1/36; B01D 15/361; B01D 15/388; C07K 2317/24; C07K 2317/21; C07K 2317/20

(57) ABSTRACT

There are provided efficient and cost-effective methods for purifying biomolecules in solution phase using stimuli-responsive protein-polymer conjugates. The protein-polymer conjugates comprise a target biomolecule-binding protein conjugated to a stimuli-responsive polymer and are reusable.

### RELATED APPLICATIONS

This application claims priority to U.S. Provisional Patent Application No. 62/057,642, filed on Sep. 30, 2014, the entire contents of which are hereby incorporated by reference.

### FIELD OF THE INVENTION

The present invention relates to the purification and separation of biomolecules. More specifically, if relates to the purification and separation of biomolecules such as proteins, polypeptides, antibodies, nucleic acids, lipids and the like, using a stimuli responsive protein-polymer conjugate that binds the desired biomolecules in solution and is then rendered insoluble by a change in conditions.

### BACKGROUND

Generally, the manufacture of biomolecules such as proteins involves the expression of the biomolecule in a host cell, followed by the purification of the biomolecule. The first step involves growing the host cell in a bioreactor to effect the expression of the biomolecule. Some examples of cell lines used for this purpose include mammalian cells such as Chinese hamster ovary (CHO) cells, bacterial cells such as E. coli cells, and insect cells. Once a biomolecule is expressed at the desired levels, it is removed from the host cell and purified. Suspended particulates, such as cells, cell fragments, and other insoluble matter are typically removed from the biomolecule-containing sample by filtration or centrifugation, resulting in a clarified fluid or supernatant containing the biomolecule of interest in solution as well as other soluble impurities.

The step of purification generally includes removing impurities such as host cell proteins (HCP), endotoxins, viruses, protein variants, protein aggregates, and other undesired biomolecules. It is desirable to obtain highly pure biomolecules in a simple and cost-efficient manner. Traditional purification methods typically include precipitation, e.g., by changing the salt concentration of a solution, and/or several chromatography steps, such as affinity and ion exchange chromatography on a solid support such as porous agarose, polymer, ceramic, or glass. Affinity chromatography enables purification of a biomolecule on the basis of biological function or individual chemical structure with high selectivity, well-suited for the isolation of a specific substance from complex biological mixtures. The sample is applied under conditions which favor its specific binding to the immobilized ligand. Unbound substances are washed away and the substance of interest can be recovered by changing the experimental conditions to those which favour its desorption. However, such traditional methods are often laborious, time-consuming, and expensive.

U.S. Pat. No. 8,362,217 relates to selectively soluble, stimuli responsive polymer capable of selectively and reversibly binding to desired biomolecules and methods of using such polymer to purify desired biomolecules in a biological material containing stream. The polymer is soluble under a certain set of process conditions such as pH, salt concentration or temperature and is rendered insoluble and precipitates out of solution upon a change in conditions. After precipitation, the biomolecule of interest is eluted from the polymer and recovered. However, the stimuli responsive polymers are capable of binding the desired biomolecules only at acidic pH and when precipitated out of solution, and binding to biomolecules is highly variable.

U.S. Pat. No. 8,263,343 relates to a method of purifying target biomolecules, such as proteins, from a liquid, comprising (a) providing at least one responsive polymer in an aqueous liquid, wherein the polymer comprises at least one hydrophobic portion; (b) contacting the aqueous liquid of (a) with the liquid comprising the target(s); (c) applying at least one first stimulus to the mixture resulting from (b) and maintaining it until a reversible phase separation is obtained, wherein one phase is a polymer-rich phase which comprises at least one target and the other phase is a polymer-poor phase; and (d) maintaining said stimulus, or, alternatively, applying at least one second stimulus, until the polymer-rich phase has transformed into a non-aqueous phase or a substantially solid phase; and. (e) isolating the substantially solid phase comprising the target(s). However, the binding properties of the responsive polymers to target biomolecules varies widely.

U.S. Pat. No. 7,786,213 relates to methods for preparing biomacromolecule-polymer complexes using chemical polymerization initiated by, and proceeding from, a protein, for therapeutic uses, use as intermediates for forming other materials, or use in diagnostic sensors. Polymerization can be initiated by a protein in the absence of additional initiation agents to form a protein-polymer conjugate. Alternatively, polymerization is initiated in the presence of an additional initiation agent that does not interact with the protein. However, the described protein-polymer conjugates are not responsive to changes in pH or other protonating stimuli and are not suitable for use in purification of certain biomacromolecules such as antibodies.

As more and more therapeutic strategies involving the use of biomolecules such as monoclonal human antibodies become available, and as production methods continue to yield increased amounts of biomolecule, there is a need for alternative materials and methods for the purification of such biomolecules that have sufficient capacity to bind and purify large amounts of product efficiently and cost-effectively.

### SUMMARY

There are provided herein methods for purifying biomolecules using stimuli-responsive protein-polymer conjugates which overcome at least some of the disadvantages of the prior art. In contrast to solid-state purification methods currently in use, methods provided herein are solution-based, e.g., aqueous phase-based, providing several potential advantages over methods in the art.

In one aspect, there is provided a stimuli-responsive protein-polymer conjugate for purifying a target biomolecule. Stimuli-responsive protein-polymer conjugates comprise one or more target biomolecule-binding protein conjugated to a stimuli-responsive polymer. A target biomolecule may be, for example, a protein, a peptide, a nucleic acid, a virus, a cell, a cellular organelle, a polysaccharide, a liposaccharide, a lipid, or a carbohydrate. In some embodiments, the target biomolecule is biotinylated, and the protein-polymer conjugate comprises streptavidin or avidin. In other embodiments, the target biomolecule is an antibody or a fragment thereof, and the protein-polymer conjugate comprises an antibody-binding protein such as Protein A or a functional equivalent thereof.

In another aspect, there is provided a method for purification of a target biomolecule from a biological sample. Methods comprise the steps of: a) contacting the biological sample with a protein-polymer conjugate, and allowing the protein-polymer conjugate to bind the target biomolecule, forming a bound conjugate-target biomolecule complex; b) changing a stimulus condition of the sample to precipitate the bound conjugate-target biomolecule complex; c) recovering the precipitate; d) resolubilizing the precipitated bound conjugate-target biomolecule complex and dissociating the target biomolecule therefrom; and e) removing the protein-polymer conjugate to obtain purified target biomolecule.

In some embodiments, the target biomolecule is a protein, a peptide, a nucleic acid, a virus, a cell, a cellular organelle, a polysaccharide, a liposaccharide, a lipid, or a carbohydrate. A biological sample may be, for example, a cell culture supernatant, a cell culture extract, a fermentation broth, a lysate, a monoclonal ascites fluid, a polyclonal antiserum, a mammalian cell culture or cell culture extract, etc.

Protein-polymer conjugates specifically bind a target biomolecule and are stimuli-responsive, e.g., responsive to pH. In some embodiments, a protein-polymer conjugate can bind to a target biomolecule without affecting or disrupting the pH sensitivity of the polymer or conjugate. In some embodiments, protein-polymer conjugates are sensitive to basic pH conditions, i.e., solubility is decreased by an increase in pH. In some embodiments, protein-polymer conjugates bind to biomolecule with high capacity or stoichiometry of binding, e.g., more than 1, more than 2, more than 3, or more than 4 biomolecule molecules can bind to one conjugate molecule. In some embodiments, capacity or stoichiometry of binding is higher than that obtained in solid-state systems, i.e., where the target biomolecule binding protein is affixed to a resin or other solid support. In some embodiments, protein-polymer conjugates are reusable, i.e., a protein-polymer conjugate can be reused in many biomolecule purification procedures, often without significant loss in biomolecule-binding capacity. In some embodiments, protein-polymer conjugates bind to biomolecule more strongly, i.e., at higher affinity, under separation conditions (e.g., high pH), preventing biomolecule loss during separation.

In one embodiment, there is provided a protein-polymer conjugate for isolation and/or separation of antibodies, and a method for purifying antibodies using the protein-polymer conjugate. In such embodiments, methods provided herein may have the advantage of providing a single platform that can be used for a wide range of target antibodies and fragments thereof, without having to adjust multiple system parameters such as pH, ionic strength, concentrations, and/or polymer length/size. In some embodiments, a further advantage of methods provided herein is that target antibody loss is prevented through phase separation at high pH, in contrast to other polymer systems which rely on decreases in pH to drive phase separation, and would lead to product loss through weakened binding between, e.g., Protein A and IgG. In some embodiments, yet another advantage is binding to target biomolecule at neutral pH and phases separation/precipitation at moderately basic pH, favouring or strengthening the binding of IgG to Protein A and improving pulldown efficiency.

Methods provided herein can have several uses in the field of bioseparation. In an aspect, methods according to the invention are useful to separate one or more target biomolecules from a sample, e.g., from a solution or a liquid. In another aspect, methods are used to obtain a sample from which one or more undesired biomolecules has been removed. In yet another aspect, methods may be used to concentrate one or more target biomolecules in a sample. In another aspect, methods are useful to obtain a target biomolecule in pure or substantially pure form, i.e., to purify a target biomolecule from a sample containing undesired contaminants or impurities.

In some embodiments, methods provided herein may have one or more of the following advantages: improved yield, due in some embodiments to a higher binding capacity for a target biomolecule, e.g., antibody, in solution compared to in solid-state; improved efficiency, due in some embodiments to a simpler process, with fewer required steps, and/or to ability to reuse materials; and reduced cost, due in some embodiments to ability to reuse materials, particularly the protein-polymer conjugate, and/or to ability to use less material, due in some embodiments to higher binding capacity of the material for the target biomolecule in solution. In some embodiments, an advantage of purification methods provided herein is the ability to reuse or recycle a protein-polymer conjugate for subsequent rounds of binding and purification, in contrast to chromatography resins that lose binding capacity due to harsh cleaning and re-charging conditions.

Thus in some embodiments, methods provided herein have the advantage of being suitable for large-scale operation, economical, and/or reusable.

In a further aspect, the present invention encompasses a kit for purification of biomolecules, which kit comprises a protein-polymer conjugate as described herein, optionally one or more buffers, and written instructions for purification of biomolecules from a biological sample using the protein-polymer conjugate.

Further embodiments and advantages of the present invention will appear from the appended claims and the description below.

### BRIEF DESCRIPTION OF THE DRAWINGS

For a better understanding of the invention and to show more clearly how it may be carried into effect, reference will now be made by way of example to the accompanying drawings, which illustrate aspects and features according to embodiments of the present invention, and in which:

FIG. 1 shows that Poly-DEAEMA and poly DEAEMA-Protein A demonstrate pH sensitive phase change. In (A), there is shown a 50 mg/ml solution of the reaction product of an anionic polymerization of DEAEMA, prepared in HCl (left panel); NaOH was added dropwise to the solution until cloudpoint was reached and solid-phase precipitate was seen (middle panel); HCl was then added back to the solution to demonstrate reversible phase change of polymer (right panel). In (B), there is shown a graph of UV spectroscopy showing absorbance at various pHs of solution of conjugate generated through ATRP of poly-DEAEMA initiated from modified Protein A.

FIG. 2 shows a schematic drawing illustrating one embodiment of purification methods of the invention, wherein steps 1-12 are numbered as indicated.

FIG. 3 shows a schematic drawing illustrating one embodiment of purification methods of the invention, wherein steps 1-10 are numbered as indicated.

FIG. 4 shows a graph of UV-vis spectroscopy showing normalized absorbance at various pHs of solution of protein A conjugates generated through ATRP, initiated from modified protein A, of DEAEMA, DIPAEMA, and combinations thereof (50/50, 25/75, and 75/25, as indicated). The graph shows that pH modulation is obtained by using poly DIPAEMA or a combination of DEAEMA and DIPAEMA to obtain a poly DEAEMA-co-DIPAEMA.

FIG. 5 shows a graph of zeta potential measured at various pH for poly DIPAEMA-protein A and poly DEAEMA-protein A conjugates. The isoelectric point of each conjugate in phosphate buffered saline is indicated by an arrow. The graph shows the pH influence on zeta potential of the conjugates.

### DETAILED DESCRIPTION

There are provided stimuli-responsive protein-polymer conjugates comprising a target biomolecule-binding protein conjugated to a stimuli-responsive polymer, for use in bioseparation.

The term “conjugate” is used herein to indicate that the protein and the polymer are covalently bound or linked together. A “covalent bond” is a form of chemical linkage that is characterized by the sharing of pairs of electrons between atoms, or between atoms and other covalent bonds.

A “target biomolecule” is any organic compound or macromolecule for which purification from a biological sample is desired. Non-limiting examples of target biomolecules include proteins, such as antibodies; peptides, such as oligopeptides or polypeptides; nucleic acids, such as DNA, e.g., plasmid DNA, RNA, or mononucleotides, oligonucleotides or polynucleotides; viruses, such as RNA viruses or DNA viruses; cells, such as prokaryotic or eukaryotic cells; cell organelles; polysaccharides; liposaccharides; lipids; and carbohydrates. It should be understood that the term “biomolecule” also includes any fragment or derivative, such as a recombinant or fusion product, thereof. In an embodiment, the target biomolecule is a protein, e.g., an antibody, such as a monoclonal or polyclonal antibody, e.g., of human or animal origin.

As used herein, the term “ligand” means molecules or compounds capable of interaction with target biomolecules.

A “biological sample” is typically a solution comprising the target biomolecule, i.e., a liquid comprising the target biomolecule, typically an aqueous liquid such as water or a suitable buffer. A biological sample may be at physiological conditions. In some embodiments, if a target biomolecule is produced in cell culture, e.g., in a cellular expression system, or by fermentation, then a biological sample may be a cell culture supernatant, a cell culture extract, a fermentation broth, or a lysate, etc. In some embodiments, where the target biomolecule is an antibody, the biological sample may be a monoclonal ascites fluid, a polyclonal antiserum, a mammalian cell culture, etc.

As used herein, the term “purification” includes separation and isolation from a biological sample such as a cell culture extract, a cell culture supernatant, a monoclonal ascites fluid, or a polyclonal antiserum, etc. Generally, undesired contaminants or impurities such as host cell proteins (HCPs), endotoxins, protein variants, protein aggregates and/or other, undesired biomolecules (e.g., viruses, nucleic acids, lipids where a protein is being purified) are removed from a purified biomolecule preparation.

As used herein, the term “eluent” is used in its conventional meaning in this field, i.e., to mean a buffer of suitable pH and/or ionic strength to release one or more compounds from a separation matrix or from a bound complex.

The terms “antibody” and “immunoglobulin” are used interchangeably herein and include fragments thereof that retain the binding specificity of the antibody.

A protein-polymer conjugate may be made using conventional methods, of which many are known in the art. In some embodiments, the protein-polymer conjugate is made using Atom-transfer Radical Polymerization (ATRP). For example, a conjugate may be prepared by modifying the target biomolecule-binding protein with an ATRP initiator, and reacting the protein-ATRP initiator with a monomer in the presence of a catalyst. Such methods are described, for example, in U.S. Pat. No. 7,786,213, the entire contents of which are hereby incorporated by reference. The desired polymer chain length can be obtained by varying the reaction time accordingly. Other polymerization and conjugation methods are also known and may be used to prepare protein-polymer conjugates of the invention.

As used herein, the term “stimuli responsive protein-polymer conjugate” means a protein-polymer conjugate that is selectively soluble, i.e., soluble under a certain set of conditions such as pH, salt concentration, or temperature and rendered insoluble upon a change in conditions (pH, salt concentration, or temperature). Thus, a stimuli responsive conjugate is capable of being selectively solubilized in a liquid under certain conditions and to be insoluble and to precipitate out of solution under different conditions in that liquid. This process is generally reversible, i.e., a stimuli responsive conjugate can be re-solubilized by a return to conditions that favor its solubility. A “pH responsive” or “pH sensitive” conjugate sensitive to pH, i.e., reversibly soluble based on pH. A “stimulus condition” refers to a condition such as pH, salt concentration, or temperature to which a stimuli responsive conjugate is responsive.

A “target biomolecule-binding protein” is a protein that binds specifically to a target biomolecule. A protein “specifically binds” to a target biomolecule when it binds with preferential or high affinity to the target biomolecule for which it is specific but does not substantially bind or binds with only low affinity to other biomolecules. In one embodiment, a target biomolecule-binding protein has minimal or no affinity for other biomolecules. In one embodiment, a target biomolecule-binding protein does not bind or does not substantially bind undesired contaminants or impurities, such as host cell proteins (HCPs), nucleic acids, endotoxins, viruses, protein variants, and/or protein aggregates.

The terms “polymer-protein conjugate”, “protein-polymer conjugate”, “stimuli responsive polymer-protein conjugate”, “stimuli responsive protein-polymer conjugate”, and “conjugate” are used interchangeably herein. Similarly, the terms “poly DEAEMA-Protein A” and “Protein A-poly DEAEMA” are used interchangeably. Similar terms are used to describe other polymer-protein conjugates. For example, the terms Poly-DiPAEMA-Protein A/protein A-poly-DIPAEMA, poly-DiPAEMA-co-DEAEMA-Protein A/protein A-poly-DIPAEMA-co-DEAEMA, etc., are used interchangeably herein. The terms “rProtein A” and “recombinant Protein A” are also used interchangeably. The terms “target biomolecule-binding protein” and “binding protein” are also used interchangeably.

### Stimuli-Responsive Polymers

As used herein, the term “polymer” refers to a material that includes a set of macromolecules. Macromolecules included in a polymer can be the same or can differ from one another in some fashion. A macromolecule can have any of a variety of skeletal structures, and can include one or more types of monomeric units. In particular, a macromolecule can have a skeletal structure that is linear or non-linear. Examples of non-linear skeletal structures include branched skeletal structures, such those that are star branched, comb branched, or dendritic branched, and network skeletal structures. A macromolecule included in a homopolymer typically includes one type of monomeric unit, while a macromolecule included in a copolymer typically includes two or more types of monomeric units. Examples of copolymers include statistical copolymers, random copolymers, alternating copolymers, periodic copolymers, block copolymers, radial copolymers, and graft copolymers.

As can be appreciated, a polymer can be provided in a variety of forms having different molecular weights, since a molecular weight (MW) of the polymer can be dependent upon processing conditions used for forming the polymer. Accordingly, a polymer can be referred to as having a specific molecular weight or a range of molecular weights. As used herein with reference to a polymer, the term “molecular weight (MW or Mw)” can refer to a number average molecular weight, a weight average molecular weight, or a melt index of the polymer.

The present invention is based, at least in part, on the fact that certain polymers undergo changes in properties as a result of changes in their environment (stimuli). The most common polymer property to change as a result of a stimulus is solubility, and the most common stimuli relating to solubility are temperature, salt concentration, and pH, or combinations thereof. As an example, a polymer may remain in solution as long as the pH, salt level or temperature is maintained within a certain range, but it will precipitate out of solution as soon as the condition is changed outside of said range. The polymer can be resolubilized by returning to conditions that maintain solubility.

Protein-polymer conjugates of the invention comprise a polymer that is stimuli responsive, i.e., a stimuli-responsive polymer that confers stimuli-responsiveness onto the conjugate as a whole. As used herein, “stimuli-responsive polymer” means a polymer that is selectively soluble, i.e., soluble under a certain set of conditions such as pH, salt concentration, or temperature and rendered insoluble (reversibly) upon a change in conditions (pH, salt concentration, or temperature). Thus, a stimuli responsive polymer is capable of being selectively solubilized in a liquid under certain conditions and to be insoluble and to precipitate out of solution under different conditions in that liquid. The stimuli responsive polymer can be resolubilized by a return to conditions that favor its solubility. A “pH responsive” or “pH sensitive” polymer is sensitive to pH, i.e., reversibly soluble based on pH or in response to changes in pH. For example, a pH responsive polymer may be soluble at acidic pH and insoluble at basic pH, or vice-versa, or may be soluble in a bracket of pH around neutral pH and insoluble at both acidic and basic pH.

Many stimuli responsive polymers are known and may be used in the present protein-polymer conjugates, provided that they do not interfere with the binding of the target biomolecule-binding protein to its target, and are otherwise suitable for use in methods of the invention.

In some embodiments, stimuli-responsive polymers are polymers whose transition between the soluble and insoluble state is created by decreasing and/or neutralizing the net charge of the polymer molecule. The net charge can be decreased by changing the pH to neutralize the charges on the macromolecule and hence to reduce the hydrophilicity (increase the hydrophobicity) of the macromolecule. For example, copolymers of methylmethacrylate (hydrophobic part) and methacrylic acid (hydrophilic at high pH when carboxy groups are deprotonated but more hydrophobic when carboxy groups are protonated) precipitate from aqueous solutions by acidification to pH around 5, and copolymers of methyl methacrylate (hydrophobic part) with dimethylaminethyl methacrylate (hydrophilic at low pH when amino groups are protonated but more hydrophobic when amino groups are deprotonated) are soluble at low pH but precipitate in slightly alkaline conditions. Hydrophobically modified cellulose derivatives that have pending carboxy groups, for example, hydroxypropyl methyl cellulose acetate succinate are also soluble in basic conditions but precipitate in slightly acidic media.

The pH-induced precipitation of pH-sensitive polymers can be very sharp and may require a change in pH of not more than 0.2-0.3 units. The pH responsiveness of a polymer can also be modified by adding other functional groups, such as a nocharged sugar to increase hydrophobicity (resulting in precipitation at a higher pH), or a counterion, such as a low molecular weight counterion or a polymer molecule of opposite charges (a polycomplex).

In some embodiments, the net charge of a polymer molecule can be changed by bubbling CO₂ through a polymer solution, protonating the polymer. The method used to alter the net charge of a polymer molecular and hence its solubility is not meant to be particularly limited.

Examples of pH sensitive soluble polymers include but are not limited to cationic polyelectrolytes and anionic polyelectrolytes. Cationic polyelectrolytes generally have basic groups (e.g., —NH₂) and respond to acidic conditions; non-limiting examples are chitosan, polyvinylpyridines, primary amine containing polymers, secondary amine containing polymers and tertiary amine containing polymers. Anionic polyelectrolytes generally have acidic groups (e.g., —COOH, —SO₃H) and respond to basic conditions; non-limiting examples include copolymers of acrylic acid, methacrylic acid and methyl methacrylate, as well as polyacrylic acid.

In some embodiments, the polymer of the present protein-polymer conjugate comprises one or more pH-sensitive polymer such as an anionic and/or cationic polyelectrolyte. Examples of such pH-sensitive polymers include, without limitation, polymers of acrylic acid, methacrylic acid, 2-(Diethylamino)ethyl methacrylate, 2-(Diethylamino)ethyl acrylate, 2-(tert-butylamino)-ethyl methacrylate, N,N-Diethylaminoethyl Methacrylate (DEAEMA), 2-(diisopropylamino)ethyl methacrylate (DIPAEMA) and/or copolymers thereof. In an embodiment, the polymer of the present protein-polymer conjugate is an acrylic polymer, a methacrylic polymer, or a vinyl polymer. In some embodiments, the polymer in the present protein-polymer conjugate comprises a polymer that is poly[2-(Diethylamino)ethyl methacrylate], poly[2-(Diethylamino)ethyl acrylate], or poly[2-(tert-butylamino)-ethyl methacrylate]. In some embodiments, the present protein-polymer conjugate comprises a polymer that is poly[N,N-Diethylaminoethyl Methacrylate](DEAEMA).

In some embodiments, the polymer is a synthetic polymer based on a vinyl monomer, such as without limitation acrylic acid (AAc), methacrylic acid (MAAc), maleic anhydride (MAnh), and/or aminoethyl methacrylate (AEMA).

In other embodiments, the polymer is a temperature sensitive polymer such as poly(N-acryloyl-N′-propylpiperazine)[PAcrNPP], poly(N-acryloyl-N′-methylpiperazine) [PAcrNMP], and/or poly(N-acryloyl-N′-ethylpiperazine)[PAcrNEP].

In other embodiments, the polymer is a synthetic polymer such as poly(N,N-dimethylaminoethyl methacrylate) [Poly(DMEAEMA)], poly(N,N-diethylaminoethyl methacrylate) [Poly(DEAEMA)], poly(diisopropylamino)ethyl methacrylate [poly(DIPAEMA)], or poly(diisopropylamino)ethyl methacrylate-co-poly(diethylamino)ethylmethacrylate [poly(DIPAEMA-co-DEAEMA)].

In an embodiment, the polymer is a random copolymer of methacrylic acid and methacrylate (Eudragit S 100, Rohm Pharma GMBH).

In some embodiments, the polymer is a synthetic polymer based on a monofunctional acrylate monomer with a polar tertiary amine functional group. In some embodiments, the polymer is a chain of units of tertiary amines of the dimethyl aminoethyl methacrylate family.

For thermosensitive polymers, reversible solubility is generally caused by changes in the hydrophobic-hydrophilic balance of uncharged polymers that are induced by increasing temperature. Uncharged polymers are soluble in water due to hydrogen bonding with water molecules. The efficiency of hydrogen bonding lessens as temperature increases. The phase separation of a polymer occurs when the efficiency of hydrogen bonding becomes insufficient for the solubility of the macromolecule. Thus, when the temperature of an aqueous solution of a smart polymer is raised above a certain critical temperature (which is often referred to as the transition temperature, lower critical solution temperature (LCST), or cloud point), phase separation takes place. An aqueous phase that contains practically no polymer and a polymer-enriched phase are formed. Both phases can be easily separated by decanting, centrifugation, or filtration. The temperature of the phase transitions depends on the polymer concentration and molecular weight. The phase separation is completely reversible, and the thermosensitive polymer dissolves in water when the temperature is reduced below the transition temperature.

Certain polymers, such as poly(N-vinyl caprolactam), poly(N-acryloylpiperidine), poly(N-vinylisobutyramide), poly(N-isopropyl acrylamide)(NIPAAM), poly(N-substituted acrylamide) including [poly(N-isopropylacrylamide), poly(N,N′-diethylacrylamide), and poly(Nacryloyl-N′-alkylpiperazine)] and hydroxyalkylcellulose are examples of polymers that exhibit solubility changes as a result of changes in temperature. Other polymers, such as copolymers of acrylic acid and methacrylic acid, polymers and copolymers of 2 or 4-vinylpyridine and chitosan exhibit changes in solubility as a result of changes in pH or salt. Other temperature sensitive soluble polymers include functional copolymers of N-isopropylacrylamide, functionalized agarose and functionalized polyethylene oxide.

In some embodiments, the polymer used in the present protein-polymer conjugate is hydrophobic. In an embodiment, the polymer presents a predominating hydrophobic character, but also comprises one or more hydrophilic portions. Thus, at least part of the polymer should be sufficiently hydrophilic to enable the preparation of an aqueous phase comprising the polymer. In one embodiment, the polymer will shift to a more hydrophobic conformation as said one or more stimuli are applied, e.g., as the pH is changed.

In some embodiments, the pH-sensitive polymer responds to basic conditions, i.e., its solubility changes in response to an increase in pH. In an embodiment, solubility of the pH-sensitive polymer in aqueous solution is decreased at higher pH, e.g., at basic pH, at pH higher than neutral (i.e., above 7), or at pH of about 7.5 or higher, or at pH of about 8 or higher. In an embodiment, a pH-sensitive polymer precipitates at pH of about 8 or higher, at pH of about 9 or higher, at pH of about 9.5 or higher, or at pH of about 10 or higher. In some embodiments, such pH-sensitive polymers are resolubilized in response to a decrease in pH, e.g., at acidic pH, at pH lower than neutral (i.e., above 7), at pH of about 5 or lower, at pH of about 4 or lower, at pH of about 3 or lower, or at pH of about 3.5. In alternative embodiments, solubility of the pH-sensitive polymer in aqueous solution is decreased at lower pH, e.g., at pH or about 4 or lower.

In some embodiments, the polymer in the present protein-polymer conjugate comprises an infinite number of monomer units. In other embodiments, the polymer consists of a finite number of monomer units. In an embodiment, the polymer ranges in molecular weight from about 1,000 to about 1,000,000 Da, from about 1,000 to about 250,000 Da, e.g., from about 2,000 to about 30,000 Da. Thus, in one embodiment, the molecular weight of the polymer is at least about 1000 Da. In an embodiment, the molecular weight of the polymer is at least about 200 kD, at least about 400 kD, or at least about 600 kD, and/or not more than about 700 kDa, or not more than about 1,000 kDa.

In some embodiments, the molecular weight of the conjugate is at least about 100 kD. In some embodiments, the molecular weight of the conjugate is in the range of about 100 kD to about 700 kD. In some embodiments, the molecular weight of the conjugate is in the range of about 200 kD to about 400 kD. In some embodiments, the molecular weight of the conjugate is in the range of about 200 kD to about 300 kD, about 100 kD to about 400 kD, or about 100 kD to about 300 kD.

In some embodiments, the polymer comprises about 200, or more than 200, monomer units, about 300 or more monomer units, about 400 or more monomer units, about 500 or more monomer units, about 900 or more monomer units, about 1000 or more monomer units, about 1200 or more monomer units, about 1400 or more monomer units, about 1600 or more monomer units, or about 1800 or more monomer units. In some embodiments, the polymer comprises less than about 5000 monomer units, less than about 4000 monomer units, less than about 3000 monomer units, or less than about 2000 monomer units. In one embodiment, the polymer comprises more than about 200 monomer units and less than about 2000 monomer units. In an embodiment, the polymer comprises from about 400 to about 2000 monomer units, from about 900 to about 2000 monomer units, from about 1000 to about 2000 monomer units, from about 1200 to about 1900 monomer units or from about 1400 to about 1900 monomer units.

In some embodiments, the length of the polymer chain may affect functionality of the protein-polymer conjugate. For example, in some embodiments, if a polymer chain is too long or short, then binding to target biomolecule and/or stimuli-responsiveness may be impaired. In other words, target biomolecule-binding capacity and/or stimuli-responsiveness, e.g., pH-sensitivity, can be affected by polymer chain length in some embodiments. Thus, size or length of a conjugate or polymer will be selected by a skilled artisan to maximize the desired properties of the conjugate, i.e., binding to target biomolecule and stimuli-responsiveness. In some embodiments, the polymer comprises a number of monomer units sufficient to provide a conjugate with desired capacity or stoichiometry of binding to target biomolecule and/or desired stimuli-responsiveness. In some embodiments, there is provided a protein-polymer conjugate capable of both binding specifically to target biomolecule with a desired binding capacity, e.g., at a stoichiometry of higher than 1:1, and retaining stimuli-responsiveness, e.g., pH-sensitivity.

A polymer may be a synthetic polymer or a natural polymer. The polymer used in the present protein-polymer conjugate may be obtained from commercial sources, or, alternatively, the skilled person in this field can easily synthesize suitable responsive polymers from monomers using conventional methods. “Polymerization” is a process of reacting monomer molecules together in a chemical reaction to form three-dimensional networks or polymer chains. Many forms of polymerization are known, and different systems exist to categorize them, as are known in the art. In some embodiments, polymers are synthesized using Atom-transfer Radical Polymerization (ATRP).

### Methods

There are provided methods for purifying a target biomolecule from a biological sample using stimuli responsive protein-polymer conjugates.

In some embodiments, methods provided herein may be used as a single step process, or as one step in a multi-step process. In some embodiments, methods provided herein are used to isolate a target biomolecule from a solution containing the target biomolecule. In other embodiments, methods provided herein are used to purify or substantially purify a target biomolecule from a solution, removing undesired contaminants or impurities. In further embodiments, methods are used to remove an undesired biomolecule from a sample or solution. For example, it may be desired to remove an antibody such as IgG from blood or blood plasma in order to purify a desired component such as a plasma protein. Methods provided herein may also be used to purify or store biomolecules comprising a ligand, i.e., biomolecules comprising functional groups or ligands capable of specifically binding a target, such as proteins containing an affinity ligand.

In an embodiment, there is provided a method comprising the steps of: contacting a biological sample containing a target biomolecule with a protein-polymer conjugate according to the invention, and allowing the protein-polymer conjugate to bind the target biomolecule (forming a bound target biomolecule-conjugate complex), the protein-polymer conjugate being soluble in the biological sample; changing a stimulus condition to precipitate the bound target biomolecule-conjugate complex; recovering the precipitate containing the bound target biomolecule-conjugate complex; resolubilizing the precipitated target biomolecule-conjugate complex under conditions that release or dissociate the target biomolecule from the conjugate, or disrupt binding of the conjugate to the target biomolecule (e.g., changing the stimulus condition again, this time to solubilize the bound target biomolecule-conjugate complex); and removing the protein-polymer conjugate to obtain purified target biomolecule.

In some embodiments, the pH is increased to basic conditions to precipitate the bound target biomolecule-conjugate complex, and the precipitated target biomolecule-conjugate complex is resolubilized in acidic solution. In some embodiments, the conditions that resolubilize the precipitated target biomolecule-conjugate complex also disrupt target biomolecule-conjugate binding and release the target biomolecule from the target biomolecule-conjugate complex.

In one embodiment, there is provided a method for purifying a target biomolecule from a biological sample, comprising the steps of:

a) contacting the biological sample with a protein-polymer conjugate of the invention, and allowing the protein-polymer conjugate to bind the target biomolecule, forming a bound conjugate-target biomolecule complex;

b) changing the pH of the sample to precipitate the bound conjugate-target biomolecule complex;

c) recovering the precipitate;

d) resolubilizing the precipitated bound conjugate-target biomolecule complex and dissociating the target biomolecule therefrom; and

e) removing the protein-polymer conjugate to obtain purified target biomolecule.

In some embodiments, the protein-polymer conjugate is responsive to basic conditions, i.e., the pH is increased in step b) to precipitate the bound conjugate-target biomolecule complex, and acidic pH is used in step d) to resolubilize the precipitated bound conjugate-target biomolecule complex. In some embodiments, acidic pH also disrupts conjugate binding to target biomolecule so that the target biomolecule is dissociated from the bound conjugate-target biomolecule complex upon resolubilization.

It will be understood that a protein-polymer conjugate should be soluble in the biological sample from which the target biomolecule is to be purified. In some embodiments, e.g., after synthesis of a protein-polymer conjugate using ATRP, or depending on the polymer, it may be necessary or desirable to protonate the conjugate before adding it to the biological sample, to ensure or enhance the conjugate's solubility in the biological sample. For example, CO₂ may be bubbled through a solution containing the protein-polymer conjugate to protonate the conjugate and render it soluble in the biological sample.

Once the conjugate is introduced to the biological sample, it will bind the targeted biomolecule in solution. For example, a Protein A-polymer conjugate is targeted to the Fc region of monoclonal antibodies in solution, binding with high specificity. Recovery and purification of the bound conjugate-target biomolecule complex occurs by a change in condition to which the stimuli-responsive polymer is sensitive, i.e., a change in stimulus condition such as pH, temperature, or salt concentration. In some embodiments, the bound conjugate-target biomolecule complex is precipitated from solution by increasing pH, e.g., to basic pH, to pH higher than neutral (i.e., above 7), or to pH of about 8 or higher. In some embodiments, the pH of the sample containing the bound conjugate-target biomolecule complex is increased to about 8 or higher, about 9 or higher, about 9.5 or higher, or about 10 or higher. pH may be increased by adding a base, a basic buffer or a basic solution, of which many are known in the art. Non-limiting examples include sodium hydroxide (NaOH) and high pH borate buffer, e.g., borate buffer at pH 10.

In some embodiments, depending on the particular protein-polymer conjugate being used, and without wishing to be limited by theory, it is believed that the increase in pH can induce a switch in the polymer chemistry by deprotonating the polymer chain; this creates a hydrophobic structure, which can then drive a phase separation (i.e., rendering the conjugate insoluble, causing it to precipitate from solution). In some embodiments, the phase separation can occur very quickly, e.g., within seconds.

It will be understood that it is desirable that the change in stimulus condition (temperature, pH, salt concentration, etc.) used to precipitate the bound conjugate-target biomolecule complex does not adversely affect the binding of the conjugate to the target biomolecule. In some embodiments, the binding of the conjugate to the target biomolecule is unaffected by the change in stimulus condition, e.g., by an increase in pH. In some embodiments, the binding of the conjugate to the target biomolecule is increased or strengthened by the change in stimulus condition, e.g., by an increase in pH. In some embodiments, a conjugate binds to an target biomolecule at basic pH and does not bind to the target biomolecule at acidic pH, for example at pH below about 7, at pH below about 6, at pH below about 5, at pH below about 4, or at pH below about 3. In some embodiments, a conjugate does not bind to target biomolecule, i.e., a target biomolecule is dissociated from the bound conjugate-target biomolecule complex, at pH of about 3 to about 4, at about pH 3.5, or at pH of about 4 or less.

For example, the binding of IgG to Protein A is pH sensitive and increase in pH to basic conditions strengthens the binding between Protein A and IgG. Therefore, in embodiments where the antibody-binding protein is Protein A or a functional equivalent thereof and the target biomolecule is an antibody such as IgG, the method has the advantage of preventing antibody loss during the phase separation at high pH.

In some embodiments, the insoluble bound conjugate-target biomolecule complex is recovered after precipitation. For example, the solution can be centrifuged to pellet the insoluble bound conjugate-target biomolecule complex. Other similar recovery methods are known in the art and may be used. Typically, the liquid phase, e.g., the supernatant, is discarded. In some embodiments, the insoluble precipitate is washed in a high pH buffer, such as 0.1M NaOH, to remove residual proteins and contaminants derived from the liquid phase or supernatant.

Once the insoluble bound conjugate-target biomolecule complex has been recovered, the purified or substantially purified target biomolecule is obtained by resolubilizing the insoluble bound conjugate-target biomolecule complex fraction and dissociating the target biomolecule from the protein-polymer conjugate. For example, in some embodiments the insoluble fraction is re-solubilized under conditions that disrupt the target biomolecule binding to the conjugate, thereby dissociating or eluting the target biomolecule from the bound conjugate-target biomolecule complex. For example, in embodiments where the target biomolecule-binding protein is Protein A or a functional equivalent thereof and the target biomolecule is an antibody such as IgG, the insoluble fraction is resolubilized in a low pH buffer, e.g., pH 3.5 buffer, which not only solubilizes the protein-polymer conjugate but disrupts its binding to the antibody by disrupting Protein A-IgG binding.

Once the bound conjugate-target biomolecule complex has been resolubilized and dissociated, the purified target biomolecule is isolated from the solution. In some embodiments, this step is performed by removing the protein-polymer conjugate from the solution. For example, in some embodiments the solution is passed through an ion exchange column which binds the polymer chain on the protein-polymer conjugate, while allowing the target biomolecule to pass through, into a now-purified fraction. In other embodiments, a centrifugal filtration method is used to remove the protein-polymer conjugate or to separate the target biomolecule. For example, a centrifugal filter having a molecular weight cut-off such that the conjugate is retained while the target biomolecule flows through may be used, allowing collection of the purified or substantially pure target biomolecule in the flow-through. Many similar methods are known in the art and may be used to separate the target biomolecule from the protein-polymer conjugate.

Once the purified target biomolecule has been separated from the protein-polymer conjugate, the protein-polymer conjugate may be reused. In some embodiments, the protein-polymer conjugate is optionally washed with a high pH wash, such as 0.1 M NaOH, before recovery and preparation for reuse. Such a wash may necessitate resolubilization of the conjugate before reuse, e.g., using a neutral pH buffer or by bubbling in CO₂. In an embodiment, the protein-polymer conjugate is recovered off of an ion exchange column. In some embodiments, the protein-polymer conjugate is recovered using centrifugal filtration, with a filter that allows target biomolecule to pass through, while retaining conjugate. Once recovered, a protein-polymer is resolubilized, if necessary or desired. For example, a conjugate can be re-solubilized by the bubbling through of CO₂, if necessary or desired, and then re-used for further purification cycles. Thus, in some embodiments a protein-polymer conjugate is reused, i.e., recycled, in subsequent target biomolecule purification or separation procedures.

Thus in some embodiments, an advantage of methods provided herein is the ability to reuse a protein-polymer conjugate, without significant loss in target biomolecule-binding capacity, providing greater efficiency and reduced cost.

In some embodiments of methods provided herein, a protein-polymer conjugate binds a target biomolecule with a stoichiometry of binding higher than 1:1, or about 2:1, or about 3:1, or about 4:1. In some embodiments of methods provided herein, stoichiometry of binding of a protein-polymer conjugate to its target biomolecule is higher in solution than in solid-phase, i.e., than when affixed to a solid support.

It should be understood that methods described herein may be varied based on several factors, such as the particular conjugate being used, the properties of the polymer and the target biomolecule-binding protein in the conjugate, the type of biological sample, the particular target biomolecule to be purified, and so on. The skilled artisan will vary the steps and conditions as required based on such factors using common knowledge in the field.

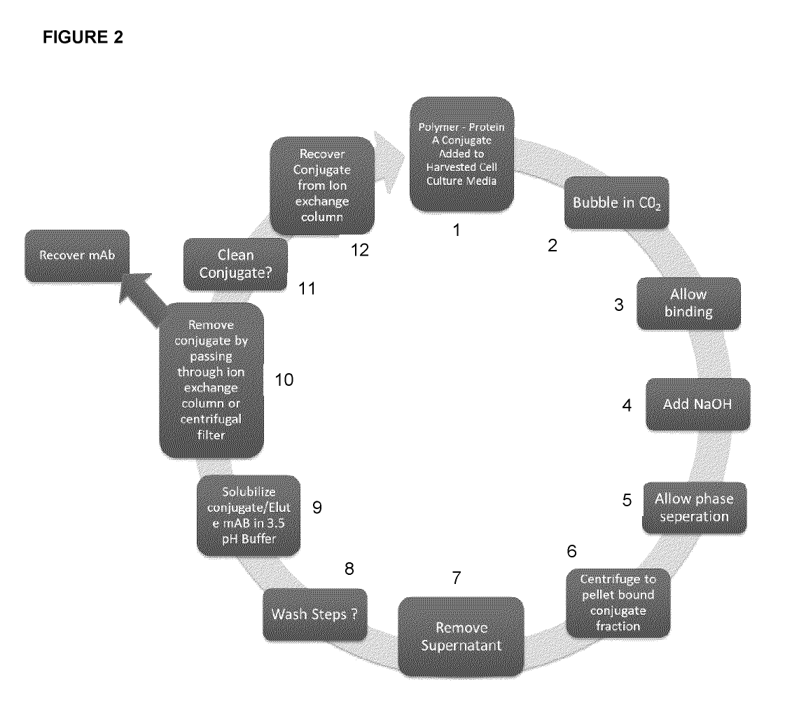

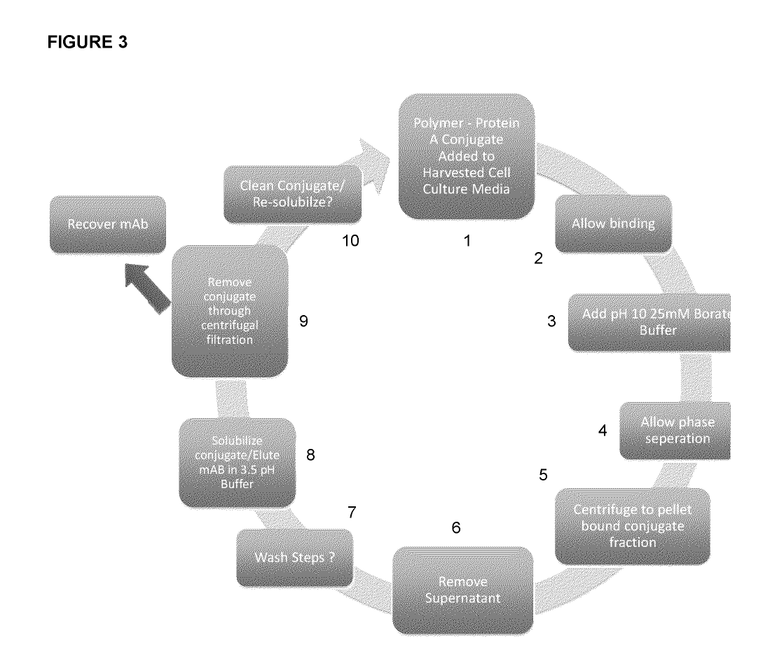

In FIGS. 2 and 3, there are shown schematic drawings illustrating exemplary embodiments of the purification methods of the invention. These drawings are meant solely to illustrate the methods of the invention and should not be construed as limiting the methods in any way.

Methods provided herein may be used to isolate target biomolecules from a variety of biological samples including, without limitation, cell culture media, culture fluids, extracts, blood, blood plasma, serum samples, ascites, and supernatants. In an embodiment, the target biomolecule-containing biological sample comprises fermentation broth. In this embodiment, the target biomolecule may be purified from host cell proteins, DNA, viruses, endotoxins, nutrients, components of a cell culture medium, such as antifoam agents and antibiotics, and/or product-related impurities, such as misfolded species and aggregates. In some embodiments, a biological sample is subjected to mechanical filtration before its contact with the protein-polymer conjugate, and consequently the sample is a clarified cell culture broth. In some embodiments, a biological sample is subjected to centrifugation before its contact with the protein-polymer conjugate to remove solids and insoluble components, and consequently the sample is a supernatant. It should be understood that target biomolecules may be recovered from a wide variety of biological samples, and the biological sample is not meant to be limited.

Methods provided herein may be used to isolate a wide range of target biomolecules. Non-limiting examples of target biomolecules include proteins, such as antibodies; peptides, such as oligopeptides or polypeptides; nucleic acids, such as DNA, e.g., plasmid DNA, RNA, or mononucleotides, oligonucleotides or polynucleotides; viruses, such as RNA viruses or DNA viruses; cells, such as prokaryotic or eukaryotic cells; cell organelles; polysaccharides; liposaccharides; lipids; and carbohydrates; as well as fragments, derivatives, variants, analogues, and functional equivalents thereof.

In some embodiments, a target biomolecule comprises a ligand or functional group capable of specifically binding a target biomolecule-binding protein. For example, in some embodiments a target biomolecule comprises biotin. In some embodiments, a target biomolecule comprises an affinity tag such as chitin binding protein, maltose binding protein, calmodulin-binding tag, or glutathione-S-transferase (GST). In some embodiments, a target biomolecule comprises an anti-epitope tag such as a V5-tag, a Myc-tag, or an HA-tag. In some embodiments, a target biomolecule comprises a fluorescent tag such as green fluorescent protein (GFP). In some embodiments, a target biomolecule comprises a metal ion that binds specifically to poly(histidine) containing fusion proteins. In some embodiments, a target biomolecule comprises a hormone or a vitamin that binds a specific receptor or carrier protein. Many site-specific DNA- and RNA-binding proteins are also known; a target biomolecule may thus include a specific DNA or RNA sequence bound by a specific binding protein included in the protein-polymer conjugate.

It should be understood that an appropriate binding protein will be selected based on the target biomolecule. For example, in embodiments where the target biomolecule comprises biotin, the protein-polymer conjugate will comprise streptavidin, avidin, or another biotin-binding protein.

### Methods for Purifying Antibodies

In a particular embodiment, the target biomolecule is an antibody or a fragment thereof, and the protein-polymer conjugate comprises an antibody-binding protein.

As used herein, the term “antibody-binding protein” refers to a protein that binds specifically to an antibody or a fragment thereof, regardless of binding mechanism. In some embodiments, an antibody-binding protein comprises an Fc-binding protein. As used herein, the term “Fc-binding protein” means a protein capable of binding to the crystallisable part (Fc) of an antibody and includes, e.g., Protein A, Protein G, Protein A/G, or a combination thereof, or a functional equivalent thereof such as a fragment or genetic derivative or fusion protein thereof that has maintained said binding property.

Protein A is a 42 kD surface protein originally found in the cell wall of the bacterium Staphylococcus aureus. It contains five high-affinity IgG-binding domains (E, D, A, B, and C) capable of interacting with the Fc region from IgG of many mammalian species such as human, mouse, and rabbit. It binds the heavy chain within the Fc region of most immunoglobulins and also within the Fab region in the case of the human VH3 family. The Z domain of Protein A is an engineered analogue of the IgG-binding domain B. Z domain contains three alpha helices which are arranged in an antiparallel three-helix bundle.

Protein G is an immunoglobulin-binding protein expressed in group C and G Streptococcal bacteria much like Protein A but with differing specificities. It is a 65 kD (G148 protein G) and a 58 kD (C40 protein G) cell surface protein that has found application in purifying antibodies through its binding to the Fab and Fc region from IgG of many mammalian species. The native molecule also binds albumin, however; because serum albumin is a major contaminant of antibody sources, the albumin binding site has been removed from many recombinant forms of Protein G used for antibody purification.

Protein A/G is a recombinant fusion protein that combines IgG binding domains of both Protein A and Protein G. For example, Protein A/G may include four Fc binding domains from Protein A and two from Protein G. Protein A/G binds to all subclasses of human IgG, making it useful for purifying polyclonal or monoclonal IgG antibodies whose subclasses have not been determined. In addition, it binds to IgA, IgE, IgM and (to a lesser extent) IgD. Protein A/G also binds to all subclasses of mouse IgG.

In some embodiments, an Fc-binding protein comprises recombinant Protein A produced in a non-mammalian source such as E. coli or insect cells. In some embodiments, an Fc-binding protein comprises recombinant Protein A produced in a mammalian source such as Chinese hamster ovary (CHO) cells. In some embodiments, an Fc-binding protein comprises recombinant Protein G produced in a non-mammalian source. In some embodiments, an Fc-binding protein comprises recombinant Protein G produced in a mammalian source. In some embodiments, an Fc-binding protein comprises recombinant Protein A/G produced in a non-mammalian source or in a mammalian source.

In some embodiments, a functional equivalent of an Fc-binding protein is used. For example, a fragment, derivative, variant, analogue, or fusion protein of Protein A or Protein G which retains the Protein A or Protein G antibody-binding properties may be used. Many recombinant or mutated forms of these Fc-binding properties are known or can be made, and it is intended that any such fragment, derivative, variant, or fusion protein of an Fc-binding protein, that retains the antibody-binding properties of the Fc-binding protein, is encompassed. “Functional equivalent” means a fragment, derivative, variant, analogue, or fusion protein of an Fc-binding protein that maintains sufficient antibody-binding affinity, specificity and/or selectivity for use in the present methods of antibody purification. The antibody-binding properties of the functional equivalent need not be identical to those of the Fc-binding protein so long as they are sufficient for use in the present methods for purification of a desired target antibody. A Fc-binding protein may also be modified to allow functionality in protein assay methods. For example, a Fc-binding protein may be biotinylated, peroxidase-conjugated, or alkaline phosphatase-conjugated for use in immunoassays (Western blot, ELISA, etc.).

In some embodiments, an Fc-binding protein comprises a monomer, dimer or multimer of Protein A domains. For example, an Fc-binding protein may comprise one or more of Domain A, B, C, D and E from Protein A. In an embodiment, an Fc-binding protein comprises one or more of Domain B and/or Domain C from Protein A. In one embodiment, an Fc-binding protein comprises Protein Z, which is a mutated form of Domain B (See, e.g., U.S. Pat. No. 5,143,844). In another embodiment, an Fc-binding protein comprises a recombinant Protein A with four IgG binding sites.

In alternative embodiments, the antibody binding protein of the present protein-polymer conjugate comprises a kappa (κ) light chain-binding protein such as Protein L or a functional equivalent thereof. In some embodiments, an Fc-binding protein comprises recombinant Protein L produced in a non-mammalian source. In some embodiments, an Fc-binding protein comprises recombinant Protein L produced in a mammalian source.

Protein L was first isolated from the surface of bacterial species Peptostreptococcus magnus and was found to bind immunoglobulins through light (L) chain interactions. Unlike Protein A and Protein G, which bind to the Fc region of immunoglobulins, Protein L binds only to antibodies that contain kappa light chains. However, since no part of the heavy chain is involved in the binding interaction, Protein L binds a wider range of antibody classes than Protein A or G. Protein L binds to representatives of all antibody classes, including IgG, IgM, IgA, IgE and IgD. Single chain variable fragments (scFv) and Fab fragments also bind to Protein L. It should be noted that Protein L is only effective in binding certain subtypes of kappa light chains. For example, it binds human VκI, VκIII and VκIV subtypes but does not bind the VκII subtype. Binding of mouse immunoglobulins is restricted to those having VκI light chains. Protein L is often used for purification of monoclonal antibodies from ascites or cell culture supernatant that are known to have the kappa light chain, and can be very useful for purification of VLκ-containing monoclonal antibodies from culture supernatant because it does not bind bovine immunoglobulins, which are often present in the media as a serum supplement.

In some embodiments, an antibody binding protein comprises a non-recombinant Fc-binding protein, i.e., a native protein isolated from a bacterial source. In one embodiment, an Fc-binding protein comprises native Protein A isolated from Staphylococcus aureus. In another embodiment, an Fc-binding protein comprises native Protein G isolated from Streptococcal bacteria.

Although Protein A, G, A/G and L all bind to mammalian immunoglobulins, it is known that their binding properties differ among species and among subclasses of IgG. For instance, Protein A binds strongly to human IgG1, IgG2 and IgG4, but not to IgG3. Protein A is sometimes preferred for rabbit, pig, dog and cat IgG, while Protein G may have better binding capacity for a broader range of mouse and human IgG subclasses (IgG1, IgG2, etc.). Protein A/G is sometimes considered to bind the broadest range of IgG subclasses from rabbit, mouse, human and other mammalian samples. Because kappa light chains occur in members of all classes of immunoglobulin (i.e., IgG, IgM, IgA, IgE and IgD), Protein L can purify these different classes of antibody. However, only those antibodies within each class that possess the appropriate kappa light chains will bind. Therefore, empirical testing may sometimes be required to determine if a particular antibody-binding protein is effective for purifying a particular antibody.

In general, many specific antibody-binding proteins are known and may be used in the present protein-polymer conjugates, provided that they do not interfere with the stimuli-responsiveness, e.g., the pH-sensitivity, of the polymer and/or the conjugate and are otherwise suitable for use in methods of the invention. A skilled artisan will select an appropriate protein for purifying a particular antibody using known techniques and available information about a protein's antibody-binding properties. Suitable methods for selecting an antibody-binding protein are well-known to those of skill in the art.

In some embodiments, an antibody-binding protein binds with high affinity and/or with specificity to IgG. In some embodiments, an antibody-binding protein binds with high affinity and/or with specificity to an antibody originating from a mammal, e.g., a mammal selected from human, mouse, rat, rodent, primate, rabbit, hamster, guinea pig, cow, sheep, goat, and pig, or to an antibody originating from a chicken. In some embodiments, an antibody-binding protein binds with high affinity and/or with specificity to an antibody originating from cultured cells such as hybridomas. In some embodiments, an antibody-binding protein binds with high affinity and/or with specificity to a human or humanized antibody. In some embodiments, an antibody-binding protein binds with high affinity and/or with specificity to a monoclonal antibody. In other embodiments, an antibody-binding protein binds with high affinity and/or with specificity to a polyclonal antibody. In some embodiments, an antibody-binding protein binds with high affinity and/or with specificity to a chimeric antibody. In some embodiments, an antibody-binding protein binds with high affinity and/or with specificity to a recombinant antibody. In some embodiments, an antibody-binding protein binds with high affinity and/or with specificity to a single-chain antibody. It should be understood that in every case an antibody-binding protein may bind to an antibody or to fragments thereof that retain the desired functionalities.

In some embodiments, an antibody-binding protein does not bind or does not substantially bind host cell proteins (HCPs). In some embodiments, an antibody-binding protein does not bind or does not substantially bind proteins in a cell culture or extract other than the desired or targeted antibody, and/or does not bind or does not substantially bind undesired contaminants, such as HCPs, nucleic acids, endotoxins, viruses, protein variants, and/or protein aggregates.

In one embodiment, the protein-polymer conjugate of the invention comprises a recombinant Protein A ligand with four IgG binding sites conjugated to a polymer chain of units of tertiary amines of the dimethyl aminoethyl methacrylate family.

Thus in some embodiments, the target biomolecule is an antibody. In such embodiments, the conjugate comprises an antibody-binding protein conjugated to a stimuli-responsive polymer.

The immune system is composed of many interdependent cell types that collectively protect the body from bacterial, parasitic, fungal, viral infections and from the growth of tumour cells. When challenged by infection or immunization, B-cells are stimulated to produce proteins called antibodies, which bind to the foreign invader. The binding event between antibody and antigen marks the foreign invader for destruction via phagocytosis or activation of the complement system. Five different classes of antibodies, or immunoglobulins, exist: IgA, IgD, IgE, IgG, and IgM. They differ not only in their physiological roles but also in their structures. From a structural point of view, IgG antibodies are a particular class of immunoglobulins that have been extensively studied, perhaps because of the dominant role they play in a mature immune response.

The biological activity of immunoglobulins is today exploited in a range of different applications in the human and veterinary diagnostic, health care and therapeutic sector. Indeed, monoclonal antibodies and recombinant antibody constructs have become the largest class of proteins currently investigated in clinical trials and receiving FDA approval as therapeutics and diagnostics.

Generally, the manufacture of antibodies involves two main steps: (1) the expression of the antibody in a host cell, followed by (2) the purification of the antibody from the cell supernatant. The first step involves growing the desired host cell in a bioreactor to effect the expression of the antibody. Some examples of cell lines used for this purpose include mammalian cells such as Chinese hamster ovary (CHO) cells, bacterial cells such as E. coli cells, and insect cells. Once the protein is expressed at the desired levels, the protein is removed from the host cell and harvested. Suspended particulates, such as cells, cell fragments, lipids and other insoluble matter are typically removed from the protein-containing sample by filtration or centrifugation, resulting in a clarified fluid or supernatant containing the antibody of interest in solution as well as other soluble impurities. Other traditional methods for isolation of immunoglobulins involve several chromatography steps such as affinity chromatography, ion exchange chromatography, and hydrophobic interaction chromatography.

For immunoglobulins (e.g., IgG), including monoclonal antibodies, Protein A is a selective affinity ligand which binds most sub-classes (See, e.g., Boyle, M. D. P. and Reis, K. J., 1987, Biotechnology, 5: 697). Protein G is another affinity ligand for IgG. (Hermanson, G. T. et al., Immobilized Affinity Ligand Techniques, Academic Press, 1992). Both Proteins A and G can bind more than one IgG. Once immobilized onto a porous chromatography support such as a resin, membrane or other media, both are useful for purification and commercial production of polyclonal IgG or monoclonal antibodies (Mabs). Protein A may be isolated in its native form from Staphylococcus aureus or recombinantly produced, e.g., in E. coli. Many modified and/or recombinant forms of Protein A have been described (See, e.g., U.S. Pat. No. 5,151,350; U.S. Pat. No. 5,084,559; U.S. Pat. No. 6,399,750; U.S. Pat. No. 7,834,158). Protein A ligands comprising a cysteine residue are also known (See, e.g., U.S. Pat. No. 5,084,559; U.S. Pat. No. 6,399,750). The addition of a cysteine amino acid facilitates ligand coupling to a base matrix or resin. Modifications to the B domain of Protein A have also been described. (See, e.g., U.S. Pat. No. 7,834,158).

Protein A and Protein G affinity chromatography are popular and widespread methods for isolation and purification of immunoglobulins, particularly for isolation of monoclonal antibodies, mainly due to the ease of use and the high purity obtained. In particular, Protein A-based affinity chromatography is widely used in industrial manufacturing of antibodies. However, existing Protein A chromatography resins are expensive and often require harsh cleaning and re-charging techniques under acidic or alkaline conditions, e.g., with sodium hydroxide (NaOH). Typically, exposure of an affinity chromatography matrix to repeated cleaning cycles results in significant loss of binding capacity of the matrix for a target molecule over time, requiring the use of a greater amount throughout the process. This is both uneconomical and undesirable as it results in the purification process becoming more expensive as well as lengthy.

Further, the binding capacity of immobilized Protein A in solid state is greatly reduced compared to the binding capacity of Protein A in free solution, requiring use of high amounts of Protein A per gram of antibody recovered. This is particularly disadvantageous in view of current production methods for monoclonal antibodies that involve fermentation of mammalian cells in bioreactors on the scale of 10,000-20,000 liters, with high titer fermentation batches that can result in total product protein amounts of over 20 Kg. The increase in total protein per batch places an increased demand on the binding capacity of the Protein A column.

Thus in an embodiment, there is provided herein a method of purifying antibodies using a protein-polymer conjugate of the invention. It is noted that the reaction kinetics and equilibrium constants for a binding reaction between an immobilized ligand and a target antibody are often very different from their behavior in free solution. Significant effects of the immobile matrix microenvironment on protein diffusion through a column, as well as inter-ligand steric hindrance, are known to reduce the binding capacities of solid-state affinity columns. Thus, the binding stoichiometry of immobilized Protein A for IgG molecules is approximately 1:1, whereas the binding stoichiometry of Protein A and IgG in free solution can reach up to 3.9. An aqueous phase-based strategy or solution-based strategy is thus expected to offer improved binding capacity.

Thus in some embodiments, there are provided herein methods of purification of antibodies from a biological sample using a solution-based process, e.g., aqueous phase-based, in contrast to solid-state chromatography methods currently in use where Protein A/antibody are bound to an immobile matrix. In some embodiments therefore, an advantage of antibody purification methods provided herein is improved yield and/or reduced cost, due at least in part to the use of a freely diffusible Protein A ligand, which means using less Protein A per gram of antibody recovered. In some embodiments, an advantage of antibody purification methods provided herein is high yield, due at least in part to use of a freely diffusible Protein A ligand, which increases the binding capacity of the Protein A (e.g., the binding stoichiometry of Protein A to IgG) compared to solid-state methods.

In an embodiment, there is provided a method comprising the steps of: contacting a biological sample containing a target antibody with a protein-polymer conjugate according to the invention, and allowing the protein-polymer conjugate to bind the target antibody (forming a bound antibody-conjugate complex), the protein-polymer conjugate being soluble in the biological sample; changing a stimulus condition to precipitate the bound antibody-conjugate complex; recovering the precipitate containing the bound antibody-conjugate complex; resolubilizing the precipitated antibody-conjugate complex under conditions that release or dissociate the antibody from the conjugate, or disrupt binding of the conjugate to the antibody (e.g., changing the stimulus condition again, this time to solubilize the bound antibody-conjugate complex); and removing the protein-polymer conjugate to obtain purified antibody. In some embodiments, the pH is increased to basic conditions to precipitate the bound antibody-conjugate complex, and the precipitated antibody-conjugate complex is resolubilized in acidic solution. In some embodiments, the conditions that resolubilize the precipitated antibody-conjugate complex also disrupt antibody-conjugate binding and release the antibody from the antibody-conjugate complex.

In one embodiment, there is provided a method for purifying an antibody from a biological sample, comprising the steps of:

a) contacting the biological sample with a protein-polymer conjugate of the invention, and allowing the protein-polymer conjugate to bind the antibody, forming a bound conjugate-antibody complex;

b) changing the pH of the sample to precipitate the bound conjugate-antibody complex;

c) recovering the precipitate;

d) resolubilizing the precipitated bound conjugate-antibody complex and dissociating the antibody therefrom; and

e) removing the protein-polymer conjugate to obtain purified antibody.

In some embodiments, the protein-polymer conjugate is responsive to basic conditions, i.e., the pH is increased in step b) to precipitate the bound conjugate-antibody complex, and acidic pH is used in step d) to resolubilize the precipitated bound conjugate-antibody complex. In some embodiments, acidic pH also disrupts conjugate binding to antibody so that the antibody is dissociated from the bound conjugate-antibody complex upon resolubilization.

It will be understood that a protein-polymer conjugate should be soluble in the biological sample from which the antibody is to be purified. In some embodiments, e.g., after synthesis of a protein-polymer conjugate using ATRP, or depending on the polymer, it may be necessary or desirable to protonate the conjugate before adding it to the biological sample, to ensure or enhance the conjugate's solubility in the biological sample. For example, CO₂ may be bubbled through a solution containing the protein-polymer conjugate to protonate the conjugate and render it soluble in the biological sample.

Once the conjugate is introduced to the biological sample, it will bind the targeted antibody in solution. For example, a Protein A-polymer conjugate is targeted to the Fc region of monoclonal antibodies in solution, binding with high specificity. Recovery and purification of the bound conjugate-antibody complex occurs by a change in condition to which the stimuli-responsive polymer is sensitive, i.e., a change in stimulus condition such as pH, temperature, or salt concentration. In some embodiments, the bound conjugate-antibody complex is precipitated from solution by increasing pH, e.g., to basic pH, to pH higher than neutral (i.e., above 7), or to pH of about 8 or higher. In some embodiments, the pH of the sample containing the bound conjugate-antibody complex is increased to about 8 or higher, about 9 or higher, about 9.5 or higher, or about 10 or higher. pH may be increased by adding a base, a basic buffer or a basic solution, of which many are known in the art. Non-limiting examples include sodium hydroxide (NaOH) and high pH borate buffer, e.g., borate buffer at pH 10.

In some embodiments, depending on the particular protein-polymer conjugate being used, and without wishing to be limited by theory, it is believed that the increase in pH can induce a switch in the polymer chemistry by deprotonating the polymer chain; this creates a hydrophobic structure, which can then drive a phase separation (i.e., rendering the conjugate insoluble, causing it to precipitate from solution). In some embodiments, the phase separation can occur very quickly, e.g., within seconds.

It will be understood that it is desirable that the change in stimulus condition (temperature, pH, salt concentration, etc.) used to precipitate the bound conjugate-antibody complex does not adversely affect the binding of the conjugate to the antibody. In some embodiments, the binding of the conjugate to the antibody is unaffected by the change in stimulus condition, e.g., by an increase in pH. In some embodiments, the binding of the conjugate to the antibody is increased or strengthened by the change in stimulus condition, e.g., by an increase in pH. In some embodiments, a conjugate binds to an antibody at basic pH and does not bind to the antibody at acidic pH, for example at pH below about 7, at pH below about 6, at pH below about 5, at pH below about 4, or at pH below about 3. In some embodiments, a conjugate does not bind to antibody, i.e., an antibody is dissociated from the bound conjugate-antibody complex, at pH of about 3 to about 4, at about pH 3.5, or at pH of about 4 or less.

For example, the binding of IgG to Protein A is pH sensitive and increase in pH to basic conditions strengthens the binding between Protein A and IgG. Therefore, in embodiments where the antibody-binding protein is Protein A or a functional equivalent thereof and the target antibody is IgG, the method has the advantage of preventing antibody loss during the phase separation at high pH.

In some embodiments, the insoluble bound conjugate-antibody complex is recovered after precipitation. For example, the solution can be centrifuged to pellet the insoluble bound conjugate-antibody complex. Other similar recovery methods are known in the art and may be used. Typically, the liquid phase, e.g., the supernatant, is discarded. In some embodiments, the insoluble precipitate is washed in a high pH buffer, such as 0.1M NaOH, to remove residual proteins and contaminants derived from the liquid phase or supernatant.

Once the insoluble bound conjugate-antibody complex has been recovered, the purified antibody is obtained by resolubilizing the insoluble bound conjugate-antibody complex fraction and dissociating the antibody from the protein-polymer conjugate. For example, in some embodiments the insoluble fraction is re-solubilized under conditions that disrupt the antibody binding to the conjugate, thereby dissociating or eluting the antibody from the bound conjugate-antibody complex. For example, in embodiments where the antibody-binding protein is Protein A or a functional equivalent thereof and the antibody is an IgG, the insoluble fraction is resolubilized in a low pH buffer, e.g., pH 3.5 buffer, which not only solubilizes the protein-polymer conjugate but disrupts its binding to the antibody by disrupting Protein A-IgG binding.

Once the bound conjugate-antibody complex has been resolubilized and dissociated, the purified antibody is isolated from the solution. In some embodiments, this step is performed by removing the protein-polymer conjugate from the solution. For example, in some embodiments the solution is passed through an ion exchange column which binds the polymer chain on the protein-polymer conjugate, while allowing the antibody to pass through, into a now-purified fraction. In other embodiments, a centrifugal filtration method is used to remove the protein-polymer conjugate or to separate the antibody. For example, a centrifugal filter or a tangential flow filtration unit having a filtration membrane with molecular weight cut-off such that the conjugate is retained while the antibody flows through may be used, allowing collection of the purified antibody in the flow-through In some embodiments, this step is performed by using a protein-polymer conjugate able to precipitate at acidic pH and removing the solid thus obtained from the solution by centrifugation of filtration. Many similar methods are known in the art and may be used to separate the antibody from the protein-polymer conjugate.

Once the purified antibody has been separated from the protein-polymer conjugate, the protein-polymer conjugate may be reused. In some embodiments, the protein-polymer conjugate is optionally washed with a high pH wash, such as 0.1 M NaOH, before recovery and preparation for reuse. Such a wash may necessitate resolubilization of the conjugate before reuse, e.g., using a neutral pH buffer or by bubbling in CO₂. In an embodiment, the protein-polymer conjugate is recovered off of an ion exchange column. In some embodiments, the protein-polymer conjugate is recovered using centrifugal filtration, with a filter that allows antibody to pass through, while retaining conjugate. Once recovered, a protein-polymer is resolubilized, if necessary or desired. For example, a conjugate can be re-solubilized by the bubbling through of CO₂, if necessary or desired, and then re-used for further purification cycles. Thus, in some embodiments a protein-polymer conjugate is reused, i.e., recycled, in subsequent antibody purification procedures.

Thus in some embodiments, an advantage of methods provided herein is the ability to reuse a protein-polymer conjugate, without significant loss in antibody-binding capacity, providing greater efficiency and reduced cost.

In some embodiments of methods provided herein, a protein-polymer conjugate binds a target antibody with a stoichiometry of binding higher than 1:1, or about 2:1, or about 3:1, or about 4:1.

It should be understood that methods described herein may be varied based on several factors, such as the particular conjugate being used, the properties of the polymer and the antibody-binding protein in the conjugate, the type of biological sample, the type of antibody to be purified, and so on. The skilled artisan will vary the steps and conditions as required based on such factors using common knowledge in the field.

In FIGS. 2 and 3, there are shown schematic drawings illustrating exemplary embodiments of the purification methods of the invention. These drawings are meant solely to illustrate the methods of the invention and should not be construed as limiting the methods in any way.

Methods provided herein may be used to purify any kind of monoclonal or polyclonal antibody without limitation, such as antibodies originating from mammalian hosts, such as mice, rodents, primates and humans, or antibodies originating from cultured cells such as hybridomas or mammalian cell expression systems. In one embodiment, the purified antibodies are human or humanized antibodies. In an embodiment, the purified antibodies are chimeric antibodies. In an embodiment, the purified antibodies are recombinant antibodies. In an embodiment, the purified antibodies are therapeutic antibodies. In an embodiment, the purified antibodies are single-chain antibodies.

In another embodiment, the purified antibodies are selected from antibodies originating from the group that consists of mouse, rat, rabbit, hamster, rodent, primate, guinea pig, cow, sheep, goat, pig, camel, and chicken. The antibodies may be of any class, i.e., selected from the group that consists of IgA, IgD, IgE, IgG, and IgM. In one embodiment, the purified antibodies are immunoglobulin G (IgG). In a specific embodiment, the IgGs are selected from the group that consists of human IgG1, human IgG2, human IgG4, human IgGA, human IgGD, human IgGE, human IgGM, mouse IgG1, mouse IgG2a, mouse IgG2b mouse IgG3, rabbit Ig, hamster Ig, guinea pig Ig, bovine Ig, and pig Ig. Thus, in one embodiment, the antibodies are monoclonal antibodies. As is well known, monoclonal antibody technology involves fusion of immortal cells, having the ability to replicate continuously, with mammalian cells to produce an antibody. The resulting cell fusion or ‘hybridoma’ will subsequently produce monoclonal antibodies in cell culture. In this context, it is to be understood that the term “antibodies” also includes antibody fragments and any fusion protein that comprises an antibody or an antibody fragment. Thus, the present method is useful to isolate any immunoglobulin-like molecule, which presents the Protein A and/or Protein G and/or Protein L binding properties of an immunoglobulin.

In other embodiments, the purified antibodies are polyclonal antibodies.

Methods provided herein may be used to isolate antibodies from a variety of biological samples including, without limitation, cell culture media, culture fluids, extracts, blood, blood plasma, serum samples, ascites, and supernatants. In an embodiment, the antibody-containing biological sample comprises fermentation broth. In this embodiment, the antibodies may be purified from host cell proteins, DNA, viruses, endotoxins, nutrients, components of a cell culture medium, such as antifoam agents and antibiotics, and/or product-related impurities, such as misfolded species and aggregates. In some embodiments, a biological sample is subjected to mechanical filtration before its contact with the protein-polymer conjugate, and consequently the sample is a clarified cell culture broth. In some embodiments, a biological sample is subjected to centrifugation before its contact with the protein-polymer conjugate to remove solids and insoluble components, and consequently the sample is a supernatant. It should be understood that monoclonal and polyclonal antibodies may be recovered from a wide variety of biological samples, and the biological sample is not meant to be limited.

In one embodiment, antibodies are purified using a protein-polymer conjugate comprising a Protein A-block-poly(diethylamino ethyl methacrylate) conjugate, wherein the Protein A is recombinant, and wherein the conjugate has a molecular weight in the range of from about 100 kD to about 400 kD or the conjugate comprises from about 400 to about 2000 DEAEMA monomer units.

### Kits

The present invention also encompasses a kit for purification of biomolecules, which kit comprises, in separate compartments, a protein-polymer conjugate as described herein, optionally one or more buffers, and written instructions for purification of biomolecules from a biological sample using the protein-polymer conjugate.

In one embodiment, the present invention encompasses a kit for purification of antibodies, which kit comprises, in separate compartments, a protein-polymer conjugate comprising an antibody-binding protein, optionally one or more buffers, and written instructions for purification of antibodies from a biological sample using the protein-polymer conjugate.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of methods described herein.

### Example 1. Preparation of a Protein A-Polymer Conjugate

### Materials.

N-(2-Hydroxyethyl)maleimide (97%), sodium borohydride (NaBH₄, ≧99%), triethylamine (≧99%), dichloromethane (anhydrous, ≧99.8%), α-bromoisobutyryl bromide (98%), acetonitrile (CH₃CN, 99.8%), petroleum ether (>95%, 30-40° C.), ethyl acetate (anhydrous, ≧99.8%), tris(2-carboxyethyl)phosphine hydrochloride (TCEP.HCl, ≧98%), sodium borohydride (≧99%), 5,5′-dithiobis(2-nitrobenzoic acid) (DTNB, ≧98%), and hydrochloride acid (34-37%) were obtained from Aldrich and used as received. rProtein A-Cys was bought from Biomedal (95%, Mw:29.87 kD). N,N-Diethylamino ethyl methacrylate (DEAEMA), N,N-diethylamino ethyl acrylate (DEAEA) and 2-(tert-butylamino)ethyl methacrylate (tBAEMA) were purchased from Sigma-Aldrich, St. Louis, Mo., and passed through a basic aluminum oxide column before use.

### Synthesis of ATRP Initiator.

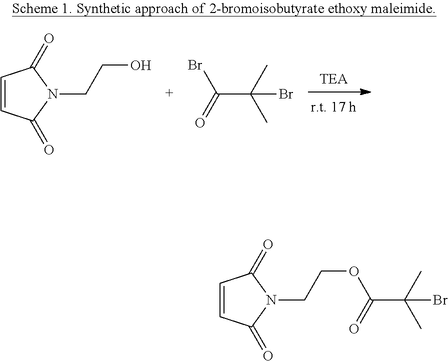

N-(2-Hydroxyethyl) maleimide (200 mg, 1.42 mmol), triethylamine (197 μL, 1.42 mmol) and dry dichloromethane (5 mL) were mixed in a 10 mL single-neck flask, which was further sealed by a septum and dipped in an ice-bath. α-Bromoisobutyryl bromide (199 μL, 1.6 mmol) was added in the mixture gradually under stirring. The reaction was run at 0° C. for two hours and then at room temperature for 15 hours. Afterward, the product was purified by silica gel chromatography using petroleum ether and ethyl acetate (4:1 v:v) as eluent. The yield of the product was 80%. ¹H NMR (300 MHz, CDCl₃, δ ppm): 6.73 (s, 2H, CH═), 4.33 (t, 2H, OCH₂), 3.86 (t, 2H, NCH₂), 1.89 (s, 6H, CH₃).

Modification of rProtein a by ATRP Initiator.

Recombinant Protein A having 4 copies of the Z binding domain and a C-terminal Cys residue (rProtein A-Cys (4×Zc) purchased from Biomedal Life Science, Seville, Spain) was used. This recombinant protein A contains 4 identical copies of an Fc region-binding domain (Z domain) assembled in a single 29.87 kD polypeptide in which the C-terminal residue has been replaced with a cysteine residue. It is produced by recombinant expression in E. coli.

The recombinant Protein A (10 mg, 3.33×10⁻⁴ mmol) was dissolved in 0.9 mL phosphate buffer (100 mM, pH=7.4). TCEP.HCl (100 μL×2.86 mg mL⁻¹, 1×10⁻³ mmol) was added in the solution under stirring. After 30 mins, 2-bromoisobutyrate ethoxy maleimide (4.87 mg, 1.66×10⁻² mmol) in 0.1 mL CH₃CN was added to the solution. The reaction was run at room temperature for 15 hours. After that, CH₃CN was removed with a vacuum rotary evaporator, and as followed, the residue was transferred into a centrifugal dialysis system (MWCO=3 kD) and dialysed against 15 mL of phosphate buffer (100 mM, pH=7.4) three times (10 000 rpm×20 min). The final concentration of rProtein A-ATRP initiator was determined from a standard curve obtained from the UV spectra of serial dilutions of rProtein A-Cys. The concentration of rProtein A-ATRP initiator was around 6 mg mL⁻¹. The measured yield of this process ranged from 37.5-66.7%.

### Testing the Modification Efficiency.

To determine the modification efficiency, the thiol group on rProtein A was titrated following attachment of the ATRP initiator by Ellman's assay (as described in Verheul, R. et al., Biomacromolecules 11, 1965-1971, 2010). Prior to the test, disulfide bonds on rProtein A owing to the cysteine-cysteine coupling reaction was reduced as follows. rProtein A (100 μL×6 mg mL⁻¹) and sodium borohydride (2 μL, 1M) were added in a 0.5 mL eppendorf tube, and incubated at 37.5° C. for 30 mins. Then hydrochloric acid (2 μL, 5M) was added into the solution to quench the reaction. The solution was transferred to a dialysis tube (MWCO=3 kD) and purified by PBS buffer (100 mM, pH=7.4) for four times (10 000 rpm×20 min). After the treatment, the concentration of rProtein A was calculated by the UV absorbance at 280 nm. Furthermore, the treated rProtein A (100 μL, 6 mg mL⁻¹) and DTNB in phosphate buffer (3 μL, 10 mM) were mixed in a 0.5 mL tube and incubated for 10 mins. The concentration of the free thiol group was calculated by UV absorbance at 412 nm. The molar concentration between rProtein A and thiol group were used to determine the modification efficiency.

Preparation of rProtein A-b-PDEAEMA Conjugate.

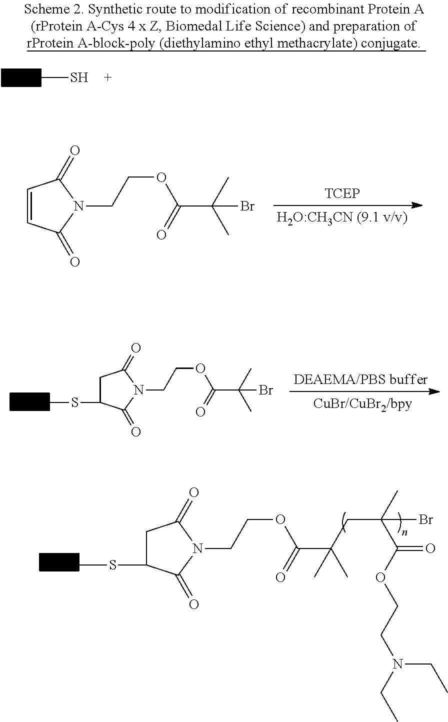

Poly DEAEMA-Protein A was prepared as follows. N,N-diethylaminoethyl methacrylate (DEAEMA) (16.8 μL, 8.35×10⁻² mmole) and rProtein A-ATRP initiator (5.0 mg, 1.67×10⁻⁴ mmole, in 300 μL 10 mM PBS buffer) were mixed in a 5 mL one-neck flask and sealed by a septum. Nitrogen was passed through the solution for 15 mins to exclude oxygen. Catalyst stock solution was prepared by mixing CuBr (13.2 mg, 9.2×10⁻² mmol), CuBr₂ (20.5 mg, 9.2×10⁻² mmol) and bpy (28.7 mg, 0.184 mmol) in 5 mL deoxygenated water. 20 μL catalyst solution was injected into the reaction solution by a syringe to start the polymerization at room temperature. 50 μL reaction solution was taken at designed times to determine the conversion of monomer by ¹H NMR. Polymer was purified twice by dissolving it in CO₂ saturated water and precipitating it from NaOH solution (pH 8.5).

A similar procedure was used to obtain other polymers and copolymers, including rProtein A-b-PDIPAEMA and rProtein A-b-PDEAEMA-co-PDIPAEMA, by replacing (diethylamino)ethylmethacrylate (DEAEMA) by either 2-(diisopropylamino)ethyl methacrylate (DIPAEMA) (8.35×10⁻² mmole) or a combination of both to obtain copolymers (50% of each, 75% DEAEMA and 25% DIPAEMA, or 25% DEAEMA and 75% DIPAEMA, for a total quantity of 8.35×10⁻² mmole).

### Example 2. Demonstration of pH Sensitivity, pH Sensitivity Modulation, and pH Influence on Polymer Zeta Potential for Polymers and Conjugates Based on Poly DEAEMA and Poly DIPAEMA

Demonstration of pH Sensitivity of Poly DEAEMA and Poly DEAEMA-Protein A Conjugate.

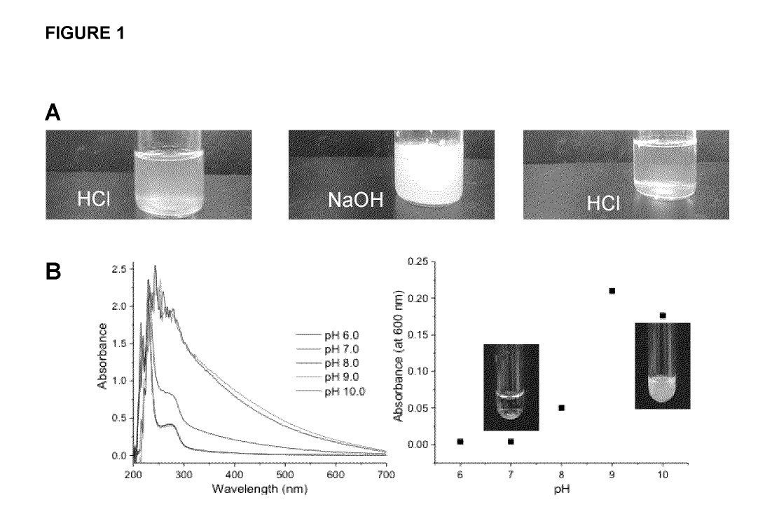

First, pH-induced phase separation of the base polymer was demonstrated. Poly-2-(diethylamino)ethyl methacrylate (poly-DEAEMA) samples were generated through an anionic polymerization of DEAEMA, as described in Example 1. The reaction products generated were then solubilized in 0.1N HCl to make a 50 mg/ml solution, which appeared clear (FIG. 1A, left panel). 0.25N NaOH was added dropwise until cloudpoint was reached and solid-phase precipitate was seen, indicating that a phase change was occurring (FIG. 1A, middle panel). Afterwards, 0.1N HCl was then added back to the solution until the solution once again was clear, demonstrating the reversible nature of the polymer's phase change (FIG. 1A, right panel).

Based on the predicted pKa and the opacity and physical appearance of the pH-induced phase, poly-2-(diethylamino)ethyl methacrylate was selected for use in conjunction with a recombinant Protein A (rProtein A-Cys 4×Z, Biomedal Life Science; also referred to herein as “rProtein A”) to generate Polymer-Protein A Conjugates using an atom transfer radical polymerization technique as described above (see Example 1). The conjugate was then solubilized in an aqueous buffer at various pHs ranging from 6.0 to 10.0 and the absorbance at 600 nm of the resulting solution was measured. UV spectroscopy was performed to measure absorbance at various pHs of solution of poly DEAEMA-Protein A conjugate generated through ATRP of poly-DEAEMA initiated from modified Protein A (as described in Example 1) (FIG. 1B). FIG. 1B shows a plot of absorbance versus wavelength (left panel) and absorbance at 600 nm versus pH (right panel). This data indicates that the conjugate demonstrated a distinct phase change response with increasing pH, particularly between pH 8 and 9. The presence of a solid phase at higher pH was confirmed visually (FIG. 1B, right panel, inset photos) and appeared white and opaque. These results indicate that the smart polymer base material provided the desired phase separation functionality in response to the pH stimulus chosen. These results also show that both poly-DEAEMA and poly DEAEMA-Protein A demonstrated pH-sensitive phase change and solubility change.

Demonstration of pH Sensitivity Modulation.

In order to modulate the pH sensitivity, the synthesis of a polymer with lower pKa was selected, namely (diisopropylamino)ethyl methacrylate (DIPAEMA). Further, modulation of the sensitivity was finely tuned by synthesizing co-polymers with different ratios of DEAEMA and DIPAEMA as described above (Example 1). The different conjugates were dissolved in an acidified phosphate saline buffer (10 mM phosphate, 2.7 mM potassium chloride, 137 mM sodium chloride, pH 5). The pH was increased by step of 0.5 upon addition of a few microliters of 0.5 M sodium hydroxide and absorbance at 600 nm was measured for each step.

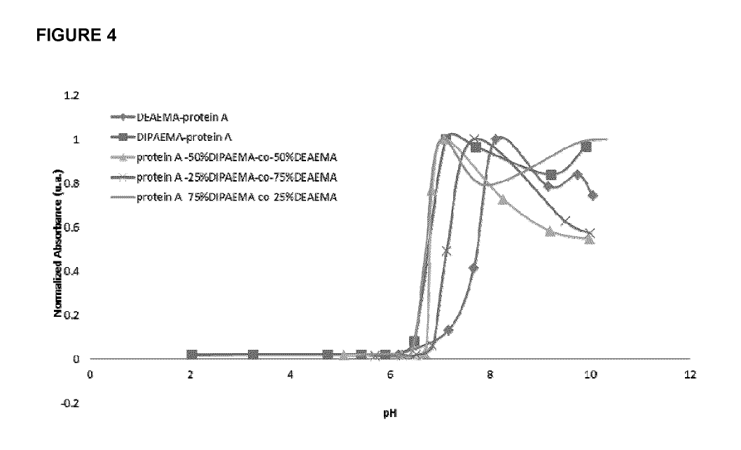

Change of polymer solubility upon pH variation was linked with a change in absorbance of the solution. UV spectroscopy was performed to measure the absorbance at various pH values for each polymer solution in order to follow the change of polymer solubility upon pH variation (FIG. 4). FIG. 4 shows a plot of absorbance at 600 nm versus pH for 2 polymers and 3 copolymers containing different ratios of DEAEMA and DIPAEMA (as described above). Each conjugate showed a clear increase of absorbance for a specific pH, indicating a solubility change of the conjugate as a response to the pH stimulus. Moreover, each conjugate presented a specific pH responsiveness which, according to theoretical pKa, was lower for conjugates rich in DIPAEMA and higher for conjugates rich in DEAEMA.

It was also seen that between pH 6.5 and 7.5 a phase change of poly-DIPAEMA and copolymers rich in DIPAEMA (75% and 50%) occurred, while between pH 7 and 8 copolymers containing 75% of DEAEMA presented a phase change, and between pH 7.5 and 9 poly-DEAEMA conjugates presented a phase change. These results indicate the possibility to not only shift pH sensitivity lower, but also to modulate the pH trigger by adjusting the ratio of DEAEMA and DIPAEMA. According to the results shown in FIG. 4, a ratio of DIPAEMA of 50% and above provided a pH sensitivity equivalent to a pure DIPAEMA conjugate, while a conjugate with 25% or less of DIPAEMA provided a pH sensitivity in-between that obtained for the DIPAEMA conjugate and DEAEMA conjugate.

Demonstration of pH Influence on Zeta Potential.

In order to see the pH influence on a conjugate's zeta potential, the synthesis of poly-(diisopropylamino)ethyl methacrylate-protein A (DIPAEMA) and poly-(diethylamino)ethylmethacrylate-protein A (DEAEMA) was performed as described above (see Example 1). The two conjugates were dissolved in an acidified phosphate saline buffer (10 mM phosphate, 2.7 mM potassium chloride, 137 mM sodium chloride, pH 5). The pH was increased by steps of 0.5 upon addition of a few microliters of 0.5 M sodium hydroxide, and zeta potential of the solution at each step was measured by a Zetasizer Nano (Malvern Instruments Ltd., Malvern, U.K.) equipped with an autotitrator.

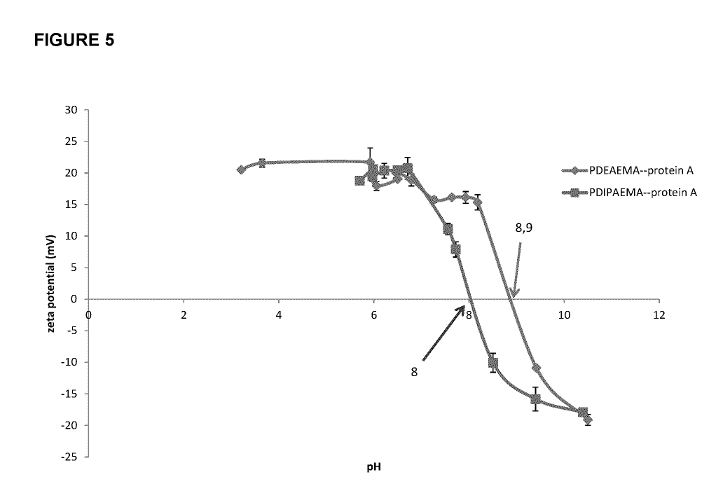

We found that change of polymer conjugate zeta potential was dependent on the polymer's isoelectric point, the buffer's ionic concentration and the pH of the solution. The Zeta potential measurement as a function of the solution's pH was performed to determine polymer isoelectric point in the presence of PBS buffer (FIG. 5). FIG. 5 shows a plot of zeta potential versus pH for two polymer-conjugates. Each conjugate showed a decrease of zeta potential from a positive to a negative value with the crossing of the abscissa axis for the pH corresponding to complete neutralization of the polymer (i.e., the isoelectric point of the conjugate). According to the theoretical pKa of each conjugate, charge neutralization of poly-DIPAEMA occurred before the neutralization of poly-DEAEMA. In this experiment the apparent isoelectric point of the poly-DIPAEMA conjugate was at pH 8 and the apparent isoelectric point of the poly-DEAEMA conjugate was at pH 8.9. These values were similarly affected by the PBS and indicated the quantity of hydroxide needed to neutralize the polymer in the presence of the PBS buffer.

Comparing FIGS. 4 and 5, it can be seen that the change in solubility of the polymers occurred before the complete neutralization of the conjugates, indicating that a small variation of a conjugate's charge was enough to strongly affect its solubility.

### Example 3. Antibody Binding Properties of Poly DEAEMA-Protein A Conjugate

Isothermal titration calorimetry is used to determine binding to an antibody, such as IgG. The unmodified rProtein A, as supplied by Biomedal, the rProtein A that was reacted with the ATRP initiator, or the polymer-Protein A conjugate are dissolved in PBS at a concentration of approx. 17 μM. 250 μL of the test solution is then added to the isothermal calorimetry (ITC) chamber and stepwise injections of limiting amounts of a purified reference IgG monoclonal antibody dissolved in PBS (78 μM) are made. The ITC apparatus (Nano ITC, TA instruments) measures the resulting change in heat that occurs upon binding of the two molecules in the chamber for each injection step and the cumulative energy change as a function of mass of the injectant. Based on the saturation point and the slope of the heat exchange curve, it is possible to calculate the thermodynamics of the binding events as well as the stoichiometry.

To eliminate the possibility that the use of the IgG is a limiting reagent in the system, creating a situation in which early injections would have far more Protein A than IgG and thus favor the binding of multiple Protein A molecules to a single IgG, ITC measurements are performed in which IgG is placed in the chamber and recombinant Protein A is used as the injectant. Binding stoichiometry in the two conditions are compared to determine if the direction of titration affects stoichiometry of bining or if stoichiometry is a property of the particular rProtein A used.

Binding stoichiometry is tested to confirm that the presence of the polymer chain does not interfere with access of IgG molecules to the binding sites of the Protein A portion of the conjugate.

These experiments are performed to confirm that the conjugate binds IgG with high affinity and equal stoichiometry to freely soluble Protein A, allowing for high efficiency recovery in solid phase. It is desirable for the polyDEAEMA-Protein A conjugate to retain the IgG binding properties of freely soluble Protein A, in particular the added binding efficiency that results from avoiding the steric hindrance effects of immobilized Protein A. These experiments also confirm that generating the polymer conjugate using ATRP initiation does not interfere with the IgG binding properties of the Protein A ligand.

Experiments are also performed to establish that the bound IgG remains bound through the pH-induced phase change and the resulting separation. In order to demonstrate this, model pull-down experiments are performed in which the polymer-Protein A conjugate is added to PBS at pH 7.0 to a concentration of 15 μM, and then measured by UV spectroscopy for absorbance at 280 nm. Afterwards a purified reference IgG monoclonal antibody is added to the solution to form a 2:1 molar ratio of conjugate to IgG, and again the absorbance at 280 nm is measured by UV spectroscopy Finally, the pH of the solution is increased by adding a pH 10.0 borate buffer (25 mM sodium borate) to induce phase separation. After centrifugation to separate the solid and liquid phases, the recovered supernatant is measured for absorbance at 280 nm

In order to quantify the percentage of unbound antibody remaining in the supernatant after pH-induced phase separation, the absorbance of the supernatant is divided by the absorbance of the initial mixture minus the absorbance of the conjugate alone. By inference, the remaining IgG is assumed to be in the precipitate, and this fraction is then referred to as the “pulldown efficiency”. This model pull-down experiment is performed using a range of pH changes to induce phase separation, and pull-down efficiencies are calculated as described above. These experiments can confirm that IgG binding to the polymer-Protein A conjugate is stable through the pH-induced phase separation and remains in the recoverable solid phase.

To confirm that the Protein A ligand is necessary for the IgG binding properties of the poly-DEAEMA-Protein A conjugate, the model pull-down experiment is repeated using unconjugated poly-2-(diethylamino)ethyl methacrylate in solution in place of the polymer-Protein A conjugate. IgG is then added. The pH is increased to 10 with high pH borate buffer (pH 10.0) and then the induced solid phase is separated out through centrifugation. These experiments can confirm that none of the IgG binds to the polymer alone and demonstrate the critical requirement for the Protein A ligand to enable the conjugate to effectively bind and separate IgG antibodies.

Another potential advantage of using a freely soluble polymer-Protein A conjugate over the use of an immobile phase is the elimination of incubation periods to allow for binding. The kinetics of binding in solution are such that, upon the introduction of the conjugate solution to an IgG antibody solution, virtually no incubation time is required. This can be demonstrated by repeating the model pull-down experiments, as described above, using increasing incubation periods before pH is increased to 10 with the addition of pH 10.0 borate buffer. When the pull-down efficiency is measured as a function of the incubation period for the binding step, the kinetics of the conjugate-to-IgG binding and the polymer phase change can be determined. It is expected that, if IgG binding to the conjugate occurs very rapidly, the increased incubation will not significantly increase pulldown efficiency.

To confirm further the optimal conditions for IgG pull-down using the pH-induced phase separation of the polymer-Protein A conjugate, the model pull-down experiments are repeated adding the IgG antibody at varying concentrations to create molar ratios of conjugate to IgG ranging from 0.5:1 to 4:1. It is determined whether the use of excess conjugate offers benefit in terms of increased pull-down of IgG and whether loss in pull-down efficiency occurs with the increase in scale.

The number and functional chemistry of the constituent monomers forming the polymer chain incorporated into the conjugate is optimized by generating a number of different samples incorporating increasing numbers of 2-(diethylamino)ethyl methacrylate monomeric units, giving rise to conjugates of increasing molecular weight, and also using increasing numbers of (tert-butylamino)-ethyl methacrylate monomers. These samples are used for a model pull-down experiment as described above, in which a 2:1 molar ratio of conjugate to IgG antibody is used and an increase in pH to 10 is used to induce phase separation. UV spectroscopy measurements of the resulting supernatants are used to determine pull-down efficiencies (Pd %) for conjugates of different chemistries between the molecular weight range of 111 kD to 372 kD, and to indicate whether and how effective separation is dependent upon the physical and chemical properties of the conjugates.

### Example 4. Screening for Host Cell Protein (HCP) Contamination of Recovered Fraction

For use as a system for the isolation and purification of antibodies, it is critical that a polymer-Protein A conjugate is able to bind antibodies selectively and accomplish phase separation without also precipitating out contaminating host cell proteins (HCP) present in harvested cell culture media from which an antibody must be separated. In order to demonstrate the purity of the recovered fraction, a model separation experiment is performed in which the various steps are examined by SDS-PAGE. Cell culture in which an antibody has been produced is analyzed on a Coomassie stained SDS-PAGE reducing gel to see if HCP is present. A 1:2 molar ratio of purified IgG and polymer-Protein A conjugate is added to the cell culture sample having target IgG added. The pH of the mixture is then raised to 10 with borate buffer and the resulting precipitate is separated by centrifugation. Samples of both the recovered supernatant and precipitate are also run on the same SDS-PAGE gel and the presence of HCP bands is determined, particularly whether they are seen in the supernatant, but absent in the precipitate, whereas the IgG band is predominantly in the precipitate, with only a small fraction remaining in the supernatant. These experiments show whether the polymer-Protein A conjugate is able to selectively purify IgG antibodies without also precipitating HCP contaminants.

### Example 5. Reusability of Recovered Polymer-Protein A Conjugate

Since the use of Protein A remains a significant cost driver for the purification of antibodies such as IgG antibodies, the economic feasibility of the described polymer-Protein A conjugate-based system depends upon the ability to recover and re-use the polymer-Protein A conjugate after the separation of the antibody. In order to demonstrate that repeated cycles of pH-induced phase separation and subsequent re-solubilization in low pH buffers does not reduce the functionality of the conjugates, a series of model pull-down experiments similar to those described above is performed. Samples of polymer-Protein A conjugate are used for IgG pull-down after being exposed to 3, 6 or 10 cycles of pH-induced phase separation at pH 10, followed by centrifugation and removal of the supernatant, and then re-solubilization in a 100 mM citrate buffer of pH 3.5. Pull-down efficiency is measured as described above and plotted as a function of cycle number. The results show whether cycles of precipitation at high pH and solubilization at low pH have significant impact on the pull-down efficiency of the conjugate, indicating whether it can be re-used in multiple purifications.

Recoverability of the polymer-Protein A conjugate through the use of centrifugal dia-filtration is demonstrated. Using a polyethersulfone based filter with a MWCO of 300 kD (from Sigma Aldrich, St. Louis, Mo.), it is confirmed that purified IgG antibody will pass through the filter when centrifuged for 2 min at 13,000 rpm. UV spectroscopy is used to measure the absorbance of the flow-through and indicates that >95% of the IgG antibody (both as provided and as a 2.3 μM solution in PBS) passes through the filter. It is tested whether conjugate passes through when a 3.8 μM solution of the polymer-Protein A conjugate in PBS is subjected to the same filtration procedure and the flow-through is measured for UV absorbance, indicating whether the filter is suitable for separating the IgG from the conjugate. Finally, a 2:1 molar ratio of polymer-Protein A conjugate:IgG antibody is prepared in a 100 mM citrate buffer of pH 3.5. These low pH conditions are similar to elution buffers used in Protein A chromatography and are predicted to interfere with IgG binding to Protein A. When this mixture is subject to the same filtration conditions and the flow-through is measured by UV spectroscopy, it can be seen what amount of IgG antibody is recovered. These experiments show whether the Poly DEAEMA-Protein A conjugate is suitable for multiple cycles of IgG binding and separation.

### Example 6. Purification of an Antibody Using a Protein-Polymer Conjugate

FIG. 2 shows a schematic drawing illustrating one embodiment of the purification methods of the invention in which a monoclonal antibody is purified. The example in this drawing is meant to illustrate the methods of the invention and should not be construed as limiting the method in any way.

In FIG. 2, step 1 (“Polymer-Protein A Conjugate Added to Harvested Cell Culture Media”) a polymer-protein conjugate is added to harvested cell culture media. For this example, a Polymer-Protein A conjugate was prepared as follows: A recombinant Protein A ligand with four IgG binding sites and a C-terminal Cys residue (rProtein A-Cys 4×Zc) was conjugated to a polymer chain of units of the tertiary amines of the dimethyl aminoethyl methacrylate family. Initially, the Protein A was modified with an ATRP (Atom-transfer Radical Polymerization) initiator (2-bromoisobutyrate ethoxy maleimide) by dissolving rProtein A-Cys in phosphate buffer (100 mM, pH=7.4). TCEP.HCl was then added in the solution under stirring. After 30 mins, the initiator in 0.1 mL CH₃CN was added to the solution. The reaction was run at room temperature for 15 hours. After that, CH₃CN was removed with a vacuum rotary evaporator, and the residue was transferred into a centrifugal dialysis system (MWCO=3 kD) and dialysed against phosphate buffer.

The conjugate was then prepared by mixing a monomer (2-(Diethylamino)ethyl methacrylate, or 2-(Diethylamino)ethyl acrylate, or 2-(tert-butylamino)-ethyl methacrylate) and the Protein A-ATRP initiator in a one-neck flask and sealed by a septum. Nitrogen was passed through the solution for 15 mins to exclude oxygen. Catalyst stock solution was prepared by mixing CuBr, CuBr₂ and bpy in deoxygenated water. The catalyst solution was injected into the reaction solution by a syringe to start the polymerization at room temperature. The reaction solution was taken at designed times to achieve conjugate of desired polymer chain length (32 kD, 105 kD, 180 kD, 250 kD, and 420 kD) and purified through dissolving in CO₂ saturated water and precipitating it from NaOH solution (pH 8.5).

The resulting conjugate can optionally be protonated through the bubbling in of CO₂ to enhance its solubility (FIG. 2, step 2). The conjugate is soluble at the pH of harvested cell culture media. By introducing the conjugate to the cell culture media, the polymer-protein A conjugate is targeted to the Fc region of monoclonal antibodies in solution, binding with high specificity and at an increased stoichiometric ratio as compared to immobilized Protein A (FIG. 2, step 3). Recovery and purification of the bound target product (conjugate bound to target antibody) occurs by a modest elevation of pH, e.g., to 8 or to 9, through the introduction of low concentration NaOH (FIG. 2, step 4) or by addition of another base, such as basic borate buffer at pH10, which induces a switch in polymer chemistry by deprotonating the polymer chain and creating a hydrophobic structure, which then drives a phase separation within minutes (FIG. 2, step 5).

Importantly, the binding of IgG to Protein A is pH sensitive and the increase in pH strengthens binding between Protein A and IgG. This prevents product loss through phase separation at high pH, in contrast to other polymer systems which rely on decreases in pH to drive phase separation, and would lead to product loss through weakened binding between Protein A and IgG in this example.

The bound, insoluble fraction produced upon elevation of the pH is recovered through centrifugation, and the supernatant is discarded (FIG. 2, steps 6 and 7). Optionally, the bound, insoluble fraction is washed with high pH buffer, such as 0.1M NaOH, to remove residual proteins derived from supernatant (FIG. 2, step 8). The antibody is then recovered by re-solubilizing the pellet fraction in a low pH buffer (e.g., pH 3.5 buffer), which not only solubilizes the polymer conjugate but elutes or dissociates the antibody by disrupting its binding to Protein A (FIG. 2, step 9).

The solution is then passed through an ion exchange column which will bind the now fully protonated polymer chain on the protein-polymer conjugate, while allowing the antibody to pass through into a now-purified fraction (FIG. 2, step 10). It should be understood that other methods known in the art may also be used to separate the antibody. For example, centrifugal filtration may be used. As an example, the solution may be passed through a centrifugal filter such as a Pall Nanosep 300 kD MWCO centrifugal filter (Sigmal Aldrich, St. Louis, Mo.), for which IgG but not conjugate passes through, allowing recovery of purified antibody in the flow-through.

The conjugate is optionally washed, e.g., in a high pH wash such as 0.1 M NaOH. This wash will induce precipitation and necessitate resolubilization, for example in neutral buffer and/or with bubbling through of CO₂ (FIG. 2, step 11). Conjugate is recovered off of the ion exchange column, if appropriate (FIG. 2, step 12). Once recovered, the protein-polymer conjugate is re-solubilized, optionally, if necessary or desired, by the bubbling through of CO₂ and re-used for further antibody-binding/purification cycles. For example, the recycled protein-polymer conjugate can be used for purifying antibody from subsequent batches of harvested cell culture fluid. Thus, in some embodiments an advantage of antibody purification methods provided herein is the ability to reuse or recycle a protein-polymer conjugate for subsequent rounds of purification, in contrast to chromatography resins that lose binding capacity due to harsh cleaning and re-charging conditions.

### Example 7. Purification of an Antibody Using a Protein-Polymer Conjugate

FIG. 3 shows a schematic drawing illustrating one embodiment of the purification methods of the invention in which a monoclonal antibody is purified. The example in this drawing is meant to illustrate the methods of the invention and should not be construed as limiting the method in any way.

In FIG. 3, step 1 (“Polymer-Protein A Conjugate Added to Harvested Cell Culture Media”) a polymer-protein conjugate is added to harvested cell culture media. For this example, a Polymer-Protein A conjugate is prepared as described above. The conjugate is soluble at the pH of the harvested cell culture media. By introducing the conjugate to the cell culture media, the polymer-protein A conjugate is targeted to the Fc region of monoclonal antibodies in solution, binding with high specificity and at an increased stoichiometric ratio as compared to immobilized Protein A (FIG. 3, step 2). Recovery and purification of the bound target product (conjugate bound to target antibody) occurs by a modest elevation of pH, e.g., by adding basic borate buffer (25 mM borate buffer, pH10), which induces a switch in polymer chemistry by deprotonating the polymer chain and creating a hydrophobic structure, which then drives a phase separation (FIG. 3, steps 3 and 4).

The bound, insoluble fraction produced upon elevation of the pH is recovered through centrifugation, and the supernatant is discarded (FIG. 3, steps 5 and 6). Optionally, the bound, insoluble fraction is washed with high pH buffer, such as 0.1M NaOH, to remove residual proteins derived from supernatant (FIG. 3, step 7). The antibody is then recovered by re-solubilizing the pellet fraction in a low pH buffer (e.g., pH 3.5 buffer), which not only solubilizes the polymer conjugate but elutes or dissociates the antibody by disrupting its binding to Protein A (FIG. 3, step 8).

The conjugate is then removed from the solution using centrifugal filtration, with a filter that retains the conjugate while allowing the antibody to pass through into a now-purified flow-through (FIG. 3, step 9). For example, a Pall Nanosep® centrifugal device with 300 kD MWCO centrifugal filter (Sigma-Aldrich, St. Louis, Mo.), or similar device, is used.

The conjugate is optionally washed, e.g., in a high pH wash such as 0.1 M NaOH, and/or re-solubilized if necessary, for example in neutral buffer and/or with bubbling through of CO₂ (FIG. 3, step 10), in preparation for reuse in further cycles of antibody binding and purification.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entireties as if each had been individually and specifically incorporated by reference herein.

What is claimed is:

1. A stimuli-responsive protein-polymer conjugate comprising one or more target biomolecule-binding protein conjugated to a stimuli-responsive polymer.

2. The protein-polymer conjugate of claim 1, wherein the target biomolecule is a protein, a peptide, a nucleic acid, a virus, a cell, a cellular organelle, a polysaccharide, a liposaccharide, a lipid, or a carbohydrate.

3. The protein-polymer conjugate of claim 1 or 2, wherein the target biomolecule-binding protein binds specifically to the target biomolecule.

4. The protein-polymer conjugate of any one of claims 1 to 3, wherein the target biomolecule is biotinylated.

5. The protein-polymer conjugate of claim 4, wherein the target biomolecule-binding protein comprises streptavidin or avidin.

6. The protein-polymer conjugate of any one of claims 1 to 5, wherein the target biomolecule is an antibody or a fragment thereof.

7. The protein-polymer conjugate of claim 6, wherein the target biomolecule-binding protein comprises an antibody-binding protein.

8. The protein-polymer conjugate of claim 7, wherein the antibody-binding protein comprises an Fc-binding protein.

9. The protein-polymer conjugate of claim 8, wherein the Fc-binding protein is Protein A or a functional equivalent thereof.

10. The protein-polymer conjugate of claim 9, wherein the Protein A is native Protein A isolated from Staphylococcus aureus.

11. The protein-polymer conjugate of claim 9, wherein the Protein A is recombinant Protein A produced in a non-mammalian source.

12. The protein-polymer conjugate of claim 9, wherein the Protein A is recombinant Protein A produced in a mammalian source.

13. The protein-polymer conjugate of claim 11 or 12 wherein the recombinant Protein A comprises a Cysteine (Cys) residue at the C-terminus.

14. The protein-polymer conjugate of any one of claims 11 to 13, wherein the recombinant Protein A comprises four IgG-binding domains.

15. The protein-polymer conjugate of any one of claims 11 to 14, wherein the recombinant Protein A comprises at least one IgG-binding domain that is a B domain or a functional equivalent thereof.

16. The protein-polymer conjugate of any one of claims 11 to 14, wherein the recombinant Protein A comprises at least one IgG-binding domain that is a Z domain.

17. The protein-polymer conjugate of any one of claims 11 to 16, wherein the non-mammalian source is E. coli.

18. The protein-polymer conjugate of any one of claims 11 to 17, wherein the recombinant Protein A comprises four Z IgG-binding domains in a single polypeptide and a Cysteine (Cys) residue at the C-terminus.

19. The protein-polymer conjugate of claim 8, wherein the Fc-binding protein is Protein G or a functional equivalent thereof.

20. The protein-polymer conjugate of claim 19, wherein the Protein G is recombinant Protein A produced in a non-mammalian source.

21. The protein-polymer conjugate of claim 20, wherein the non-mammalian source is E. coli.

22. The protein-polymer conjugate of claim 19, wherein the Protein G is recombinant Protein A produced in a mammalian source.

23. The protein-polymer conjugate of claim 8, wherein the Fc-binding protein is Protein A/G or a functional equivalent thereof.

24. The protein-polymer conjugate of claim 23, wherein the Protein A/G comprises four IgG-binding domains from Protein A and two IgG-binding domains from Protein G.

25. The protein-polymer conjugate of any one of claims 7 to 24, wherein the antibody-binding protein is specific for IgG.

26. The protein-polymer conjugate of claim 7, wherein the antibody-binding protein comprises a kappa light chain-binding protein.

27. The protein-polymer conjugate of claim 26, wherein the kappa light chain-binding protein is Protein L or a functional equivalent thereof.

28. The protein-polymer conjugate of claim 27, wherein the Protein L is recombinant Protein L produced in a non-mammalian source.

29. The protein-polymer conjugate of claim 28, wherein the non-mammalian source is E. coli.

30. The protein-polymer conjugate of claim 26, wherein the Protein L is recombinant Protein A produced in a mammalian source.

31. The protein-polymer conjugate of any one of claims 1 to 30, wherein the stimuli-responsive polymer is sensitive to pH, temperature, salt concentration, or a combination thereof.

32. The protein-polymer conjugate of claim 31, wherein the stimuli-responsive polymer is reversibly soluble based on the pH, temperature, salt concentration, or a combination thereof.

33. The protein-polymer conjugate of claim 31 or 32, wherein the stimuli-responsive polymer is pH-sensitive.

34. The protein-polymer conjugate of claim 33, wherein the pH-sensitive polymer is soluble at acidic or neutral pH and insoluble at basic pH, or wherein the pH-sensitive polymer is soluble at neutral pH and insoluble at both basic and acidic pH.

35. The protein-polymer conjugate of claim 33 or 34, wherein the pH-sensitive polymer is an anionic polyelectrolyte.

36. The protein-polymer conjugate of any one of claims 31 to 35, wherein the polymer comprises a chain of units of a tertiary amine of a dimethyl aminoethyl methacrylate.

37. The protein-polymer conjugate of any one of claims 31 to 35, wherein the polymer comprises polymers of acrylic acid, methacrylic acid, 2-(Diethylamino)ethyl methacrylate, 2-(Diethylamino)ethyl acrylate, N,N-dimethylaminoethyl methacrylate (DMEEMA), 2-(tert-butylamino)-ethyl methacrylate (tBuMAEMA), N,N-Diethylaminoethyl Methacrylate (DEAEMA), 2-(diisopropylamino)ethyl methacrylate (DIPAEMA) and/or copolymers thereof.

38. The protein-polymer conjugate of claim 37, wherein the polymer is poly[2-(Diethylamino)ethyl methacrylate], poly[2-(Diethylamino)ethyl acrylate], poly[N,N-dimethylaminoethyl methacrylate], or poly[2-(tert-butylamino)-ethyl methacrylate].

39. The protein-polymer conjugate of claim 37, wherein the polymer is poly[N,N-Diethylaminoethyl Methacrylate].

40. The protein-polymer conjugate of any one of claims 33 to 39, wherein the polymer is insoluble at pH of about 8 or higher or of about 7.5 or higher, and/or insoluble at pH of about 4 or lower.

41. The protein-polymer conjugate of claim 40, wherein the polymer is insoluble at pH of about 9 or higher, or of about 9.5 or higher.

42. The protein-polymer conjugate of claim 40, wherein the polymer is insoluble at pH of about 10 or higher.

43. The protein-polymer conjugate of any one of claims 33 to 42, wherein the polymer comprises from about 200 to about 2000 monomer units.

44. The protein-polymer conjugate of any one of claims 1 to 43, wherein the conjugate is synthesized using ATRP.

45. The protein-polymer conjugate of any one of claims 1 to 44, wherein the stoichiometry of conjugate binding to antibody is greater than about 1:1.

46. The protein-polymer conjugate of any one of claims 1 to 44, wherein the stoichiometry of conjugate binding to antibody is about 2:1, about 3:1, or about 4:1.

47. The protein-polymer conjugate of any one of claims 1 to 46, wherein the conjugate binds the target biomolecule at higher capacity in solution than in solid-phase.

48. The protein-polymer conjugate of any one of claims 1 to 47, wherein the conjugate is a rProtein A-poly DEAEMA conjugate, a rProteinA-poly DIPAEMA conjugate, or a conjugate comprising a copolymer of poly DIPAEMA-co-DEAEMA, the conjugate having a molecular weight in the range of from about 100 kDa to about 400 kDa or from about 100 kDa to about 1,000 kDa.

49. The protein-polymer conjugate of any one of claims 1 to 48, wherein the conjugate is a rProtein A-poly DEAEMA conjugate comprising from about 200 to about 2000 DEAEMA monomer units or from about 200 to about 5,000 DEAEMA monomer units.

50. A method for purifying a target biomolecule from a biological sample, comprising the steps of:
a) contacting the biological sample with the protein-polymer conjugate as defined in any one of claims 1 to 49 and allowing the protein-polymer conjugate to bind the target biomolecule, forming a bound conjugate-target biomolecule complex;
b) changing a stimulus condition of the sample to precipitate the bound conjugate-target biomolecule complex;
c) recovering the precipitate;
d) resolubilizing the precipitated bound conjugate-target biomolecule complex and dissociating the target biomolecule therefrom; and
e) removing the protein-polymer conjugate to obtain purified target biomolecule.

51. The method of claim 50, wherein the target biomolecule is a protein, a peptide, a nucleic acid, a virus, a cell, a cellular organelle, a polysaccharide, a liposaccharide, a lipid, or a carbohydrate.

52. The method of claim 50 or 51, wherein the target biomolecule is biotinylated.

53. The method of any one of claims 50 to 52, wherein the target biomolecule-binding protein comprises streptavidin or avidin.

54. The method of any one of claims 50 to 53, wherein the target biomolecule is an antibody or a fragment thereof.

55. The method of claim 54, wherein the target biomolecule-binding protein comprises an antibody-binding protein.

56. The method of any one of claims 50 to 55, wherein the stimulus condition is pH, temperature, and/or salt concentration.

57. The method of claim 56, wherein the stimulus condition is pH.

58. The method of any one of claims 50 to 57, further comprising the step of:
f) recovering the protein-polymer conjugate that was removed in step e).

59. The method of claim 58, further comprising the step of:
g) protonating the recovered protein-polymer conjugate by bubbling through CO₂ so that the protein-polymer conjugate is soluble in the biological sample and can be reused in steps a)-f).

60. The method of any one of claims 52 to 59, wherein the pH is increased to basic conditions in step b) to precipitate the bound conjugate-target biomolecule complex.

61. The method of claim 60, wherein the pH is increased to about pH 8, to about pH 9, to about pH 9.5, or to about pH 10.

62. The method of claim 60 or 61, wherein binding of the conjugate to the target biomolecule is unaffected or is increased by the increase in pH.

63. The method of claim 62, wherein the conjugate does not bind to the target biomolecule at acidic pH, or at pH about 3 to about 4, or at about pH 3.5.

64. The method of any one of claims 50 to 63, wherein the precipitate is recovered by centrifugation in step c).

65. The method of any one of claims 50 to 64, wherein the precipitated bound conjugate-target biomolecule complex is resolubilized in step d) by changing the stimulus condition or reverting the stimulus condition back to its original value in the sample.

66. The method of any one of claims 50 to 65, wherein the precipitated bound conjugate-target biomolecule complex is resolubilized in acidic solution in step d).

67. The method of claim 66, wherein the acidic solution is at about pH 3.5 or at about pH 3 to about pH 4.

68. The method of claim 66 or 67, wherein the target biomolecule is dissociated from the conjugate in the acidic solution.

69. The method of any one of claims 50 to 68, wherein the protein-polymer conjugate is removed in step e) using ion-exchange chromatography, centrifugal filtration, tangential flow filtration, or filtration of the protein-polymer conjugate precipitated at acidic pH.

70. The method of any one of claims 54 to 69, wherein the antibody is a monoclonal antibody.

71. The method of any one of claims 54 to 69, wherein the antibody is an IgG.

72. The method of claim 70 or 71, wherein the antibody is a human antibody or a humanized antibody.

73. The method of claim 70 or 71, wherein the antibody is a murine antibody.

74. The method of claim 70 or 71, wherein the antibody is a chimeric antibody or a recombinant antibody.

75. The method of any one of claims 70 to 74, wherein the biological sample comprises a cell culture extract, cell culture media, or an ascites fluid.

76. The method of any one of claims 50 to 75, wherein the target biomolecule is produced in a mammalian cell expression system.

77. The method of any one of claims 54 to 69, wherein the antibody is a polyclonal antibody.

78. The method of claim 77, wherein the biological sample comprises serum or blood.
